(19) United States
(12) Patent Application Publication
(10) Pub. No.: US 2024/0057861 A1
(43) Pub. Date: Feb. 22, 2024

(54) GRADE EVALUATION APPARATUS, OPHTHALMIC IMAGING APPARATUS, NON-TRANSITORY COMPUTER-READABLE STORAGE MEDIUM, AND GRADE EVALUATION METHOD

(71) Applicant: Topcon Corporation, Tokyo (JP)

(72) Inventor: Sho NITTA, Tokyo (JP)

(73) Assignee: Topcon Corporation, Tokyo (JP)

(21) Appl. No.: 18/369,222

(22) Filed: Sep. 18, 2023

Related U.S. Application Data
(63) Continuation of application No. PCT/JP2022/011085, filed on Mar. 11, 2022.

(30) Foreign Application Priority Data
Mar. 19, 2021 (JP) 2021-045512

(51) Int. Cl. A61B 3/10; A61B 3/00; A61B 3/12; G06T 7/00; G06T 7/11; G06T 7/13; G06T 7/162

(52) U.S. Cl. CPC A61B 3/102; A61B 3/0025; A61B 3/12; G06T 7/0012; G06T 7/11; G06T 7/13; G06T 7/162; G06T 2207/10101; G06T 2207/20192; G06T 2207/30041; G06T 2207/20081; G06T 2207/20182

(57) ABSTRACT

A grade evaluation apparatus for blood vessel distribution image includes a control device. The control device is configured to: obtain a blood vessel distribution image, the blood vessel distribution image representing a distribution of blood vessels in an eyeball obtained by OCT-angiography; calculate a plurality of evaluation values from the blood vessel distribution image based on a plurality of criteria; and calculate a grade value based on the plurality of evaluation values, the grade value indicating a grade of the blood vessel distribution image.

### CROSS-REFERENCE TO RELATED APPLICATIONS

This is a continuation application of International Application No. PCT/JP2022/011085 filed on Mar. 11, 2022 which claims priority from Japanese Patent Application No. 2021-045512 filed on Mar. 19, 2021. The entire contents of the earlier applications are incorporated herein by reference.

### TECHNICAL FIELD

The present disclosure relates to a grade evaluation apparatus, an ophthalmic imaging apparatus, a non-transitory computer-readable storage medium, and a grade evaluation method.

### BACKGROUND

In the ophthalmic field, image diagnosis plays an important role. In recent years, optical coherence tomography (OCT) has been utilized. OCT has been used not only for obtainment of B-scan images and three-dimensional images of a subject eye, but also for obtainment of C-scan images and en-face images such as shadow gram.

OCT can also be used for obtaining an image emphasizing a specific site of a subject eye or obtaining functional information. For example, OCT-angiography has recently attracted attention.

OCT-angiography is typically a technique of constructing images emphasizing blood vessels (angiographic images, angiograms, and motion contrast images), based on time-series data obtained by applying OCT to a three-dimensional region of the fundus.

According to OCT-angiography, an image representing the distribution of blood vessels (hereinafter, blood vessel distribution image) is obtained. OCT-angiography is based on images obtained by OCT. If a plurality of tomographic images of OCT are captured at the same location while being shifted in time and then the tomographic images that are shifted in time are compared, red blood cells move at locations where blood vessels exist, which changes the signal intensity. An image of blood vessels can be obtained by extracting and highlighting such portions where the signal intensity changes as blood vessels. A blood vessel distribution image obtained by OCT-angiography is, for example, an image representing the distribution of the blood vessels on the superficial of the retina.

The obtainment of the blood vessel distribution image by OCT-angiography requires a plurality of OCT images shifted in time at the same location. However, if the tracking is not successful or if the fixation by the subject eye is unstable, the quality (grade) of the blood vessel distribution image obtained by OCT-angiography deteriorates. The evaluation of the grade of the blood vessel distribution image has been visually performed by an ophthalmologist in related art. However, the grade of the blood vessel distribution image cannot be evaluated quantitatively. There are a plurality of criteria for evaluating the grade of the blood vessel distribution image, but the grade of the blood vessel distribution image cannot be quantitatively evaluated based on a plurality of criteria.

JP2017-006179A describes an OCT signal processing apparatus for processing an OCT signal detected by an OCT device based on measurement light scanned on the subject and reference light. The OCT signal processing apparatus includes a control unit for causing a display unit to display a confirmation screen for confirming the quality of three-dimensional motion contrast data obtained by processing a plurality of OCT signals at different times at the same site. The control unit displays, on the confirmation screen, motion contrast images based on depth region data, which is three-dimensional motion contrast data extracted in a partial depth region of the subject.

The technique described in JP2017-006179A causes the display unit to display the confirmation screen so that a person can confirm the quality of the three-dimensional motion contrast data, rather than quantitatively evaluate the grade of the blood vessel distribution image by an apparatus.

JP2017-046976A describes an ophthalmic imaging apparatus for imaging a subject eye. The ophthalmic imaging apparatus includes a first obtainment unit for obtaining a plurality of OCT signals at different times at the same position on the subject eye by an OCT optical system for obtaining tomographic image data on the subject eye. The ophthalmic imaging apparatus includes an image processing unit for processing the OCT signals in the depth direction at each scanning position obtained by the first obtainment unit to obtain en-face motion contrast data or three-dimensional motion contrast data on the subject eye. The ophthalmic imaging apparatus includes an image dividing unit for dividing the en-face motion contrast data or the three-dimensional motion contrast data into a plurality of regions. The ophthalmic imaging apparatus includes a determination unit for determining, for each of the divided regions divided by the image dividing unit, whether the en-face motion contrast data on the divided region or the three-dimensional motion contrast data on the divided region is appropriate.

The technique described in JP2017-046976A determines whether the en-face motion contrast data on each divided region or the three-dimensional motion contrast data on each divided region is appropriate for the divided region, re-obtains a plurality of OCT signals by the second obtainment unit at each scanning position in the same divided regions as the en-face motion contrast data on the divided regions determined to be inappropriate or the three-dimensional motion contrast data on the divided regions determined to be inappropriate, and sets the re-obtained OCT signals at the scanning positions as a plurality of OCT signals in the divided regions determined to be inappropriate. That is, in view of the occurrence of positional deviation or motion artifacts in an image due to the influence of blinking or flicks of the subject eye, the technique described in JP2017-046976A divides regions and re-obtains OCT signals for the divided regions determined to be inappropriate, thereby obtaining en-face motion contrast data useful for diagnosis or three-dimensional motion contrast data useful for diagnosis. The technique described in JP2017-046976A does not quantitatively evaluate the grade of the blood vessel distribution image based on a plurality of criteria.

### SUMMARY

Illustrative aspects of the present disclosure provide a grade evaluation apparatus, an ophthalmic imaging apparatus, a non-transitory computer-readable storage medium, and a grade evaluation method quantitatively evaluating the grade of a blood vessel distribution image obtained by OCT-angiography based on a plurality of criteria.

One illustrative aspect of the present disclosure provides a grade evaluation apparatus for blood vessel distribution image, including a control device configured to: obtain a blood vessel distribution image, the blood vessel distribution image representing a distribution of blood vessels in an eyeball obtained by OCT-angiography; calculate a plurality of evaluation values from the blood vessel distribution image based on a plurality of criteria; and calculate a grade value based on the plurality of evaluation values, the grade value indicating a grade of the blood vessel distribution image.

Another illustrative aspect of the present disclosure provides an ophthalmic imaging apparatus for executing optical coherence tomography (OCT), including the grade evaluation apparatus (500) according to the aspect above.

Still another illustrative aspect of the present disclosure provides a non-transitory computer-readable storage medium storing a computer program readable by a computer, the computer program, when executed by the computer, causing the computer to perform operations including: obtaining a blood vessel distribution image, the blood vessel distribution image representing a distribution of blood vessels in an eyeball obtained by OCT-angiography; calculating a plurality of evaluation values from the blood vessel distribution image based on a plurality of criteria; and calculating a grade value based on the plurality of evaluation values, the grade value indicating a grade of the blood vessel distribution image.

Still another illustrative aspect of the present disclosure provides a grade evaluation method for blood vessel distribution image by a computer including a processor, the grade evaluation method including causing the processor to perform: calculating a plurality of evaluation values based on a plurality of criteria from a blood vessel distribution image, the blood vessel distribution image representing a distribution of blood vessels in an eyeball obtained by OCT-angiography; and calculating a grade value based on the plurality of evaluation values, the grade value indicating a grade of the blood vessel distribution image. According to the present disclosure, it is to possible to provide the grade evaluation apparatus, the ophthalmic imaging apparatus, the non-transitory computer-readable storage medium, and the grade evaluation method quantitatively evaluating the grade of a blood vessel distribution image obtained by OCT-angiography based on a plurality of criteria.

### BRIEF DESCRIPTION OF DRAWINGS

Illustrative aspects of the disclosure are illustrated by way of example and not by limitation in the accompanying figures in which like reference characters indicate similar elements.

FIG. 1 is a schematic diagram illustrating an example of the configuration of an ophthalmic imaging apparatus.

FIG. 2 is a schematic diagram illustrating an example of the configuration of the ophthalmic imaging apparatus.

FIG. 3 is a schematic diagram illustrating an example of the configuration of the ophthalmic imaging apparatus.

FIG. 4A is a schematic diagram illustrating an example of a scan pattern executable by the ophthalmic imaging apparatus.

FIG. 4B is a schematic diagram illustrating an example of a scan pattern executable by the ophthalmic imaging apparatus.

FIG. 5 is a schematic diagram illustrating an example of the configuration of a grade evaluation apparatus according to an illustrative embodiment.

FIG. 6 is a flowchart illustrating an example of the operation of the grade evaluation apparatus according to the illustrative embodiment.

FIG. 7 illustrates a blood vessel distribution image when the grade of the blood vessel distribution image is Poor.

FIG. 8 is a diagram illustrating a blood vessel distribution image when the grade of the blood vessel distribution image is Average.

FIG. 9 is a diagram illustrating a blood vessel distribution image when the grade of the blood vessel distribution image is Good.

FIG. 10 is a schematic diagram of a first algorithm.

FIG. 11 is a schematic diagram of the first algorithm.

FIG. 12 is a schematic diagram of the first algorithm.

FIG. 13 is a schematic diagram of a second algorithm.

FIG. 14 is a schematic diagram of the second algorithm.

FIG. 15 is a schematic diagram of the second algorithm.

FIG. 16 is a schematic diagram of a third algorithm.

FIG. 17 is a schematic diagram of the third algorithm.

FIG. 18 is a schematic diagram of the third algorithm.

FIG. 19 is a schematic diagram of the third algorithm.

FIG. 20 is a schematic diagram of a fourth algorithm.

FIG. 21 is a schematic diagram of the fourth algorithm.

FIG. 22 is a schematic diagram of a fifth algorithm.

FIG. 23 is a schematic diagram of the fifth algorithm.

FIG. 24 is a schematic diagram of the fifth algorithm.

FIG. 25 is a schematic diagram of the fifth algorithm.

FIG. 26 is a schematic diagram of a sixth algorithm.

FIG. 27 is a schematic diagram of the sixth algorithm.

FIG. 28 is a schematic diagram of the sixth algorithm.

FIG. 29 is a schematic diagram of the sixth algorithm.

FIG. 30 is a schematic diagram of the sixth algorithm.

FIG. 31 is a schematic diagram of a seventh algorithm.

FIG. 32 is a schematic diagram of the seventh algorithm.

FIG. 33 is a schematic diagram of an eighth algorithm.

FIG. 34 is a conceptual diagram illustrating a decision tree used by the grade evaluation apparatus according to the illustrative embodiment.

### DETAILED DESCRIPTION

Several illustrative embodiments of the present disclosure will be described in detail with reference to the drawings. The ophthalmic imaging apparatus related to the illustrative embodiments is at least an ophthalmic apparatus having a function of performing optical coherence tomography (OCT).

Hereinafter, an ophthalmic imaging apparatus in which swept source OCT and fundus camera are combined will be described, but the ophthalmic imaging apparatus is not limited thereto. For example, the type of OCT is not limited to swept source OCT, and may be spectral domain OCT or the like. Here, swept source OCT is a technique of dividing light from a variable-wavelength light source (wavelength sweeping light source) into measurement light and reference light, generating interference light by superimposing the return light of the measurement light from an object on the reference light, detecting the interference light by a balanced photodiode or the like, and performing Fourier transform or the like on detection data collected according to wavelength sweeping and scanning of the measurement light to form an image. On the other hand, spectral domain OCT is a technique of dividing light from a low-coherence light source into measurement light and reference light, generating interference light by superimposing the return light of the measurement light from an object on the reference light, detecting the spectral distribution of the interference light by a spectroscope, and performing Fourier transform or the like on the detected spectral distribution to form an image. In other words, swept source OCT is a technique of obtaining a spectral distribution by time division, while spectral domain OCT is a technique of obtaining a spectral distribution by spatial division. The method for OCT applicable to the illustrative embodiment is not limited thereto, and any other method (for example, time domain OCT) can be applied.

The ophthalmic imaging apparatus may or may not have a function of obtaining a photograph (digital photograph) of a subject eye such as a fundus camera. Instead of a fundus camera, any modality such as scanning laser ophthalmoscopy (SLO), a slit lamp microscope, an anterior segment imaging camera, or a surgical microscope may be provided. An en-face image such as a fundus photograph can be used for observation of the fundus, setting of a scan region, tracking, and the like.

{Configuration Example of Ophthalmic Imaging Apparatus}

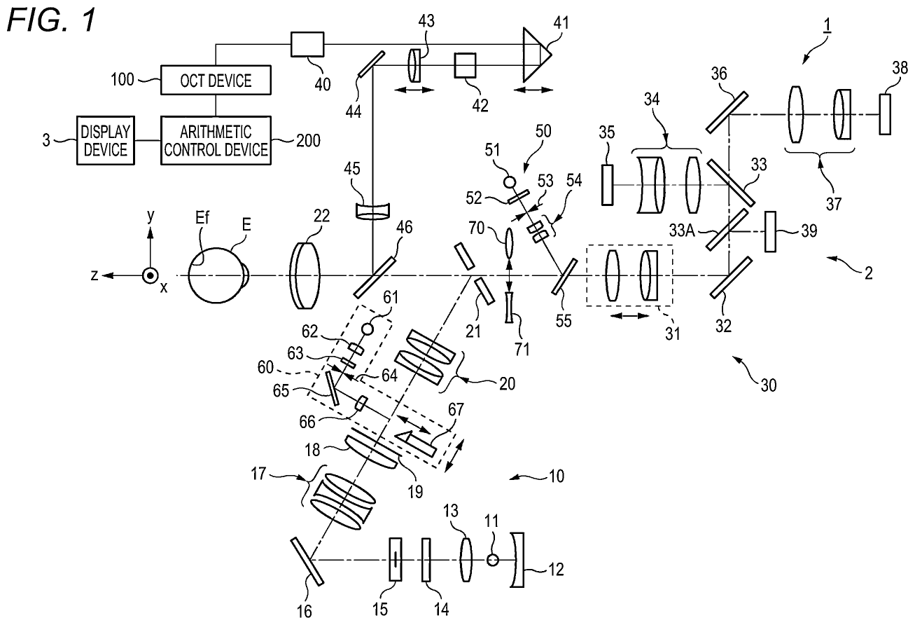

As illustrated in FIG. 1, the ophthalmic imaging apparatus 1 includes a fundus camera device 2, an OCT device 100, and an arithmetic control device 200. The fundus camera device 2 is provided with substantially the same optical system as a fundus camera in related art. The OCT device 100 is provided with an optical system and a mechanism for performing OCT. The arithmetic control device 200 includes a processor. A chin rest and a face rest for supporting the face of the subject are provided at positions facing the fundus camera device 2.

In this specification, “processor” means a circuit such as a central processing unit (CPU), a graphic processing unit (GPU), an application specific integrated circuit (ASIC), a programmable logic device (for example, a simple programmable logic device (SPLD), a complex programmable logic device (CPLD), or a field programmable gate array (FPGA). For example, the processor implements the functions according to the illustrative embodiments by reading and executing a program stored in storage circuitry or a storage apparatus.

{Fundus Camera Device 2} The fundus camera device 2 is provided with an optical system and/or a mechanism for imaging the fundus Ef of the subject eye E. Images obtained by imaging the fundus oculi Ef (referred to as a fundus image, a fundus photograph, or the like) include observation images and captured images. An observation image is obtained by, for example, video imaging using near infrared light. The captured image is, for example, a color image or a monochrome image obtained using visible flash light, or a monochrome image obtained using near-infrared flash light. The fundus camera device 2 may be capable of obtaining a fluorescein fluorescent image, an indocyanine green fluorescent image, an autofluorescence image, or the like.

The fundus camera device 2 includes an illumination optical system 10 and an imaging optical system 30. The illumination optical system 10 irradiates the subject eye E with illumination light. The imaging optical system 30 detects the return light of the illumination light from the subject eye E. The measurement light from the OCT device 100 is guided to the subject eye E through an optical path in the fundus camera device 2, and the return light is guided to the OCT device 100 through the same optical path.

An observation light source 11 of the illumination optical system 10 is, for example, a halogen lamp or a light-emitting diode (LED). The light output from the observation light source 11 (observation illumination light) is reflected by a reflection mirror 12 having a curved reflection surface, passes through the condensing lens 13, passes through a visible light cut filter 14, and becomes near infrared light. Then, the observation illumination light once converges in the vicinity of an imaging light source 15, reflected by a mirror 16, and passes through relay lenses 17 and 18, a diaphragm 19, and a relay lens 20. The observation illumination light is reflected by the peripheral portion of an apertured mirror 21 (the region around the aperture portion), passes through a dichroic mirror 46, and is refracted by an objective lens 22 to illuminate the subject eye E (particularly, the fundus Ef).

The return light of the observation illumination light from the subject eye E is refracted by the objective lens 22, passes through the dichroic mirror 46, passes through the hole formed in the central region of the apertured mirror 21, passes through a dichroic mirror 55, passes through an imaging focusing lens 31, and is reflected by a mirror 32. Then, the return light passes through a half mirror 33A, and is reflected by a dichroic mirror 33 and focused on the light detection surface of a CCD image sensor 35 by a condensing lens 34. The CCD image sensor 35 detects the return light at a predetermined frame rate, for example. When the imaging optical system 30 focuses on the fundus Ef, an observation image of the fundus Ef is obtained. When the imaging optical system 30 focuses on the anterior segment, an observation image of the anterior segment is obtained.

The imaging light source 15 is, for example, a visible light source including a xenon lamp or an LED. The light (imaging illumination light) output from the imaging light source 15 is applied to the fundus Ef through the same path as the observation illumination light. The return light of the imaging illumination light from the subject eye E is guided to the dichroic mirror 33 through the same path as the return light of the observation illumination light, transmitted through the dichroic mirror 33, reflected by the mirror 36, and focused on the light detection surface of the CCD image sensor 38 by the condensing lens 37.

The liquid crystal display (LCD) 39 displays a fixation target for fixing the subject eye E. The light flux (fixation light flux) output from an LCD 39 is partially reflected by the half mirror 33A, reflected by the mirror 32, passes through the imaging focusing lens 31 and the dichroic mirror 55, and passes through the aperture of the apertured mirror 21. The fixation light flux having passed through the aperture of the apertured mirror 21 passes through the dichroic mirror 46, and is refracted by the objective lens 22 and projected onto the fundus Ef. The fixation position of the subject eye E can be changed by changing the display position of the fixation target on the screen of the LCD 39. Instead of the LCD 39, a matrix LED in which a plurality of LEDs are two-dimensionally arranged, a combination of a light source and a variable diaphragm (liquid crystal diaphragm or the like), or the like can be used as a fixation light flux generator.

The fundus camera device 2 is provided with an alignment optical system 50 and a focus optical system 60. The alignment optical system 50 generates an alignment index used for alignment of the optical system with respect to the subject eye E. The focus optical system 60 generates a split indicator used for focus adjustment for the subject eye E.

The alignment light output from the LED 51 of the alignment optical system 50 passes through diaphragms 52 and 53 and a relay lens 54, is reflected by the dichroic mirror 55, and passes through the aperture of the apertured mirror 21. The light passing through the aperture of the apertured mirror 21 passes through the dichroic mirror 46 and is projected onto the subject eye E through the objective lens 22.

The corneal reflection light of the alignment light passes through the objective lens 22, the dichroic mirror 46, and the hole, and partially passes through the dichroic mirror 55, passes through the imaging focusing lens 31, is reflected by the mirror 32, passes through the half mirror 33A, and is reflected by the dichroic mirror 33 and projected onto the light detection surface of the CCD image sensor 35 by the condensing lens 34. Manual alignment and automatic alignment similar to those in the related art can be performed based on the image detected by the CCD image sensor 35 (alignment index image including two bright points).

The focus optical system 60 is moved along the optical path (illumination optical path) of the illumination optical system 10 in conjunction with the movement of the imaging focusing lens 31 along the optical path (imaging optical path) of the imaging optical system 30. A reflection rod 67 is insertable into and removal from the illumination optical path.

When the focus adjustment is performed, the reflection surface of the reflection rod 67 is inclined in the illumination optical path. The focus light output from the LED 61 passes through the relay lens 62, is separated into two light fluxes by the split indicator plate 63, passes through the two-hole diaphragm 64, is reflected by the mirror 65, and is once focused on the reflection surface of the reflection rod 67 by the condensing lens 66 to be reflected. Further, the focus light passes through the relay lens 20, and is reflected by the apertured mirror 21, transmitted through the dichroic mirror 46, refracted by the objective lens 22, and projected onto the fundus Ef.

The fundus reflection light of the focus light is detected by the CCD image sensor 35 through the same path as the corneal reflection light of the alignment light. Manual alignment and automatic alignment similar to those in the related art can be performed based on the image detected by the CCD image sensor 35 (split indicator image including two bright line images).

The imaging optical system 30 includes visibility correction lenses 70 and 71. The visibility correction lenses 70 and 71 can be selectively inserted into the imaging optical path between the apertured mirror 21 and the dichroic mirror 55. The visibility correction lens 70 is a plus (+) lens for correcting high hyperopia, and is, for example, a convex lens of +20 D (diopter). The visibility correction lens 71 is a negative (−) lens for correcting high myopia, and is, for example, a −20 D concave lens. The visibility correction lenses 70 and 71 are attached to, for example, a turret plate. The turret plate is formed with a hole for a case where neither of the visibility correction lenses 70 and 71 is applied.

The dichroic mirror 46 combines an optical path for fundus imaging and an optical path for OCT. The dichroic mirror 46 reflects light in a wavelength band used for OCT and transmits light for fundus imaging. A collimator lens device 40, an optical path length changer 41, an optical scanner 42, an OCT focusing lens 43, a mirror 44, and a relay lens 45 are provided in the optical path for OCT in order from the OCT device 100.

The optical path length changer 41 is movable in the direction of the arrow illustrated in FIG. 1 to change the optical path length of the OCT optical path. The change of the optical path length is used for correction of the optical path length according to the axial length of the subject eye E, adjustment of the interference state, and the like. The optical path length changer 41 includes, for example, a corner cube and a mechanism for moving the corner cube.

The optical scanner 42 is arranged at a position optically conjugate with the pupil of the subject eye E. The optical scanner 42 changes the traveling direction of the measurement light LS passing through the optical path for OCT. Accordingly, the subject eye E is scanned with the measurement light LS. The optical scanner 42 can deflect the measurement light LS in any direction in the xy plane, and includes, for example, a galvanometer mirror for deflecting the measurement light LS in the x direction and a galvanometer mirror for deflecting the measurement light LS in the y direction.

{OCT Device 100}

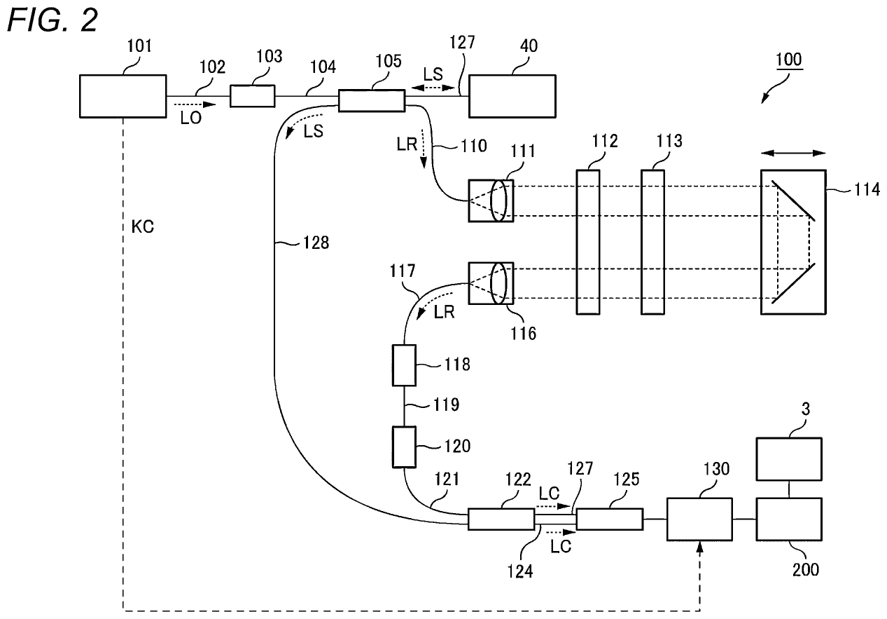

As illustrated in FIG. 2, the OCT device 100 is provided with an optical system for performing OCT of the subject eye E. The configuration of this optical system is similar to that of a swept source OCT in the related art. That is, the optical system includes an interference optical system for dividing light from a light source into measurement light and reference light, superimposing the return light of the measurement light from the subject eye E and the reference light having passed through the reference optical path to generate interference light, and detecting the interference light. The detection result (detection signal) obtained by the interference optical system is a signal indicating the spectrum of the interference light, and is sent to the arithmetic control device 200.

A light source device 101 includes a variable-wavelength light source capable of fast changing the wavelength of the emitted light, similarly to a general swept source OCT. The variable-wavelength light source is, for example, a near-infrared laser light source.

Light L0 output from the light source device 101 is guided to a polarization controller 103 by the optical fiber 102, so that the polarization state thereof is adjusted. Then, the light L0 is guided to a fiber coupler 105 by an optical fiber 104 to be divided into measurement light LS and reference light LR.

The reference light LR is guided to the collimator 111 by an optical fiber 110 to be converted into a parallel light flux, and is guided to a corner cube 114 via an optical path length correcting member 112 and a dispersion compensating member 113. The optical path length correcting member 112 operates to match the optical path length of the reference light LR and the optical path length of the measurement light LS. The dispersion compensating member 113 operates to match the dispersion characteristics between the reference light LR and the measurement light LS.

The corner cube 114 turns the traveling direction of the incident reference light LR to the opposite direction. The incident direction and the emission direction of the reference light LR with respect to the corner cube 114 are parallel to each other. The corner cube 114 is movable in the incident direction of the reference light LR, and the optical path length of the reference light LR changes accordingly.

The configuration illustrated in FIGS. 1 and 2 is provided with both the optical path length changer 41 for changing the length of the optical path (measurement optical path, measurement arm) of the measurement light LS and the corner cube 114 for changing the length of the optical path (reference optical path, reference arm) of the reference light LR, but may be provided with one of the optical path length changer 41 and the corner cube 114 alone. It is also possible to change the difference between the measurement optical path length and the reference optical path length by using other optical members.

The reference light LR having passed through the corner cube 114 passes through the dispersion compensating member 113 and the optical path length correcting member 112, and is converted from a parallel light flux into a converging light flux by a collimator 116 and incident on an optical fiber 117. The reference light LR incident on the optical fiber 117 is guided to a polarization controller 118 so that the polarization state thereof is adjusted, guided to an attenuator 120 by an optical fiber 119 so that its light amount is adjusted, and guided to a fiber coupler 122 by an optical fiber 121.

On the other hand, the measurement light LS generated by the fiber coupler 105 is guided by an optical fiber 127, converted into a parallel light flux by the collimator lens device 40, passes through the optical path length changer 41, the optical scanner 42, the OCT focusing lens 43, the mirror 44, and the relay lens 45, reflected by the dichroic mirror 46, refracted by the objective lens 22, and incident on the subject eye E. The measurement light LS is scattered and reflected at various depth positions of the subject eye E. The return light of the measurement light LS from the subject eye E travels in the same path as the outward path in the reverse direction, is guided to the fiber coupler 105, and reaches the fiber coupler 122 via the optical fiber 128.

The fiber coupler 122 generates interference light by superimposing the measurement light LS incident through the optical fiber 128 and the reference light LR incident through the optical fiber 121. The fiber coupler 122 generates a pair of interference light LC by splitting the interference light at a predetermined splitting ratio (for example, 1:1). The pair of interference lights LC are guided to the detector 125 through optical fibers 123 and 124, respectively.

The detector 125 is, for example, a balanced photodiode. The balanced photodiode includes a pair of photodetectors for respectively detecting the pair of interference lights LC, and outputs the difference between the detection results obtained by the photodetectors. The detector 125 sends the detection result (detection signal) to a data acquisition system (DAQ) 130.

The DAQ 130 is supplied with a clock KC from the light source device 101. The clock KC is generated in the light source device 101 in synchronization with the output timing of each wavelength swept within a predetermined wavelength range by the variable-wavelength light source. For example, the light source device 101 optically delays one of the two split lights obtained by splitting the light L0 of each output wavelength, combines the spilt lights again, and then generates the clock KC based on a result of detecting the combined light. The DAQ 130 samples the detection signal input from the detector 125 based on the clock KC. The DAQ 130 sends the sampling result of the detection signal from the detector 125 to the arithmetic control device 200.

{Arithmetic Control Device 200}

The arithmetic control device 200 is configured to control each component of the fundus camera device 2, the display apparatus 3, and the OCT device 100. Further, the arithmetic control device 200 executes various arithmetic processing. For example, the arithmetic control device 200 performs signal processing such as Fourier transform on a spectral distribution based on the detection result obtained by the detector 125 for each series of wavelength scans (for each A-line), thereby forming a reflection intensity profile for each A-line. Further, the arithmetic control device 200 forms image data by converting the reflection intensity profile of each A-line into images. The arithmetic processing for this purpose is similar to that of a swept source OCT in the related art.

The arithmetic control device 200 includes, for example, a processor, a RAM (random access memory), a ROM (read only memory), a hard disk drive, and a communication interface. Various computer programs are stored in a storage apparatus such as the hard disk drive. The arithmetic control device 200 may include an operation device, an input device, a display device, and the like.

{Control System}

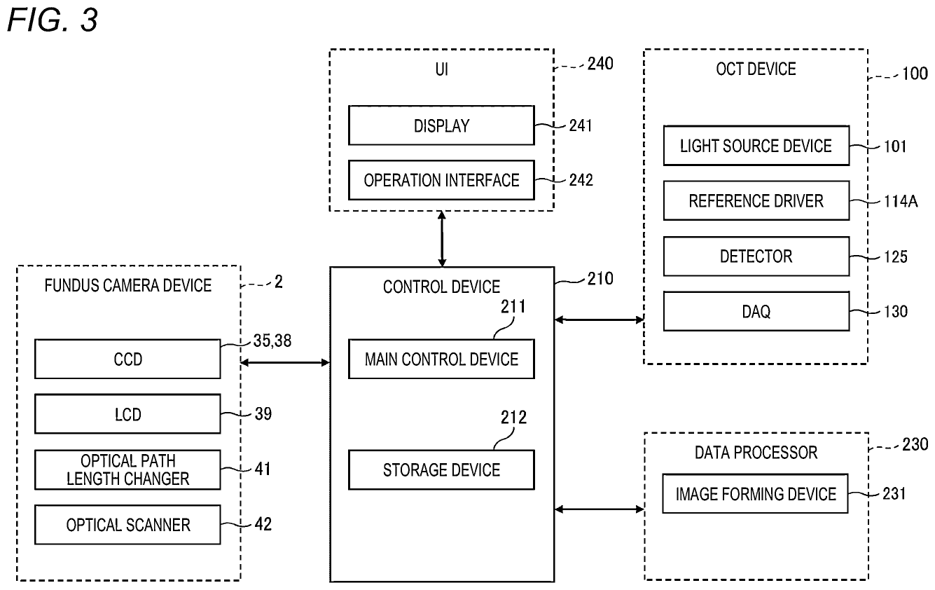

FIG. 3 illustrates a configuration example of a control system (processing system) of the ophthalmic imaging apparatus 1. {Control Device 210}

The control device 210 controls each component of the ophthalmic imaging apparatus 1. The control device 210 includes a processor, a RAM, a ROM, a hard disk drive, and the like. The function of the control device 210 is implemented by cooperation between hardware including circuitry and control software. The control device 210 includes a main control device 211 and a storage device 212.

{Main Control Device 211}

The main control device 211 performs various controls. For example, the main control device 211 controls the imaging focusing lens 31, the CCD image sensor 35, the CCD image sensor 38, the LCD 39, the optical path length changer 41, the optical scanner 42, the OCT focusing lens 43, the focus optical system 60, the reflection rod 67, the light source device 101, a reference driver 114A, the detector 125, the DAQ 130, and the like. The reference driver 114A moves the corner cube 114 provided in the reference optical path. Accordingly, the length of the reference optical path is changed. The ophthalmic imaging apparatus 1 may include a user interface (UI) 240 having a display 241 and an operation interface 242. The main control device 211 may perform various controls in accordance with a user's operation received through the operation interface 242 and may display various information on the display 241.

{Storage Device 212}

The storage device 212 stores various data. Examples of the data stored in the storage device 212 include image data on an OCT image, image data on a fundus image, and subject eye information. The subject eye information includes subject information such as the patient ID and the name, identification information indicating the left/right eye, electronic medical record information, and the like.

{Data Processor 230}

The data processor 230 executes various data processing. For example, the data processor 230 forms image data based on data collected by the DAQ 130. The data processor 230 can apply processing (signal processing) on the data collected by the DAQ 130, apply image processing or analysis processing on the image data formed from the data collected by the DAQ 130, and apply image processing or analysis processing on the image data output from the CCD image sensor 35 (or the CCD image sensor 38). The data processor 230 includes, for example, at least one of a processor and a dedicated circuit board. The data processor 230 includes an image forming device 231.

{Image Forming Device 231}

The image forming device 231 includes an image forming processor (not illustrated). The image forming device 231 forms cross-sectional image data on the fundus oculi Ef based on the sampling result of the detection signal input from the DAQ 130 to the data processor 230. This processing includes signal processing such as noise removal (noise reduction), filter processing, and fast Fourier transform (FFT) as in a swept source OCT in the related art. The image data formed by the image forming device 231 is a data set including a group of image data formed by converting the reflection intensity profiles on a plurality of A-lines (scan lines along the z-direction) arranged along the scan lines (a group of A-scan image data).

When OCT-angiography is performed, the image forming device 231 can form a motion contrast image based on detection data (for example, a detection signal group from the DAQ 130) collected by scans repeatedly performed a predetermined number of times. This motion contrast image is an angiographic image (angiogram) emphasizing blood vessels of the fundus oculi Ef. The motion contrast image is an image created based on a plurality of data (images) obtained at the same position at different times, and is an image representing the motion at the position.

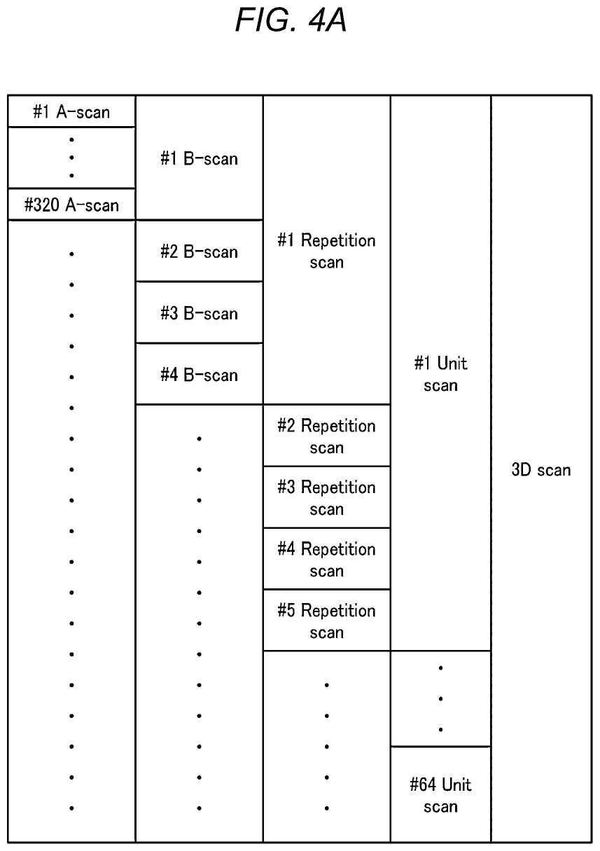

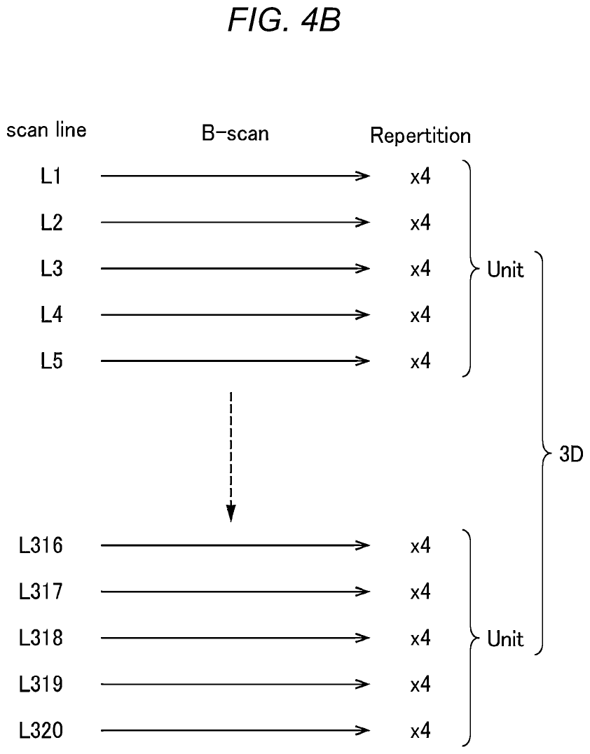

Here, a typical example of a scan pattern applicable to OCT-angiography will be described. In OCT-angiography, three-dimensional scan (raster scan) is applied. The three-dimensional scan is a scan along a plurality of scan lines arranged parallel to each other. The plurality of scan lines are ordered in advance, and the scan is applied in this order. FIGS. 4A and 4B illustrate examples of three-dimensional scanning applicable to the present illustrative embodiment.

As illustrated in FIG. 4B, the three-dimensional scan of the present example is executed for 320 scan lines L1 to L320. One scan along one scan line Li (i=1 to 320) is called a B-scan. One B-scan includes 320 A-scans (see FIG. 4A). Each A-scan is a scan for one A-line. That is, each A-scan is a scan for an A-line along the incident direction of the measurement light LS (z direction, depth direction, axial direction). The B-scan includes 320 A-scans arranged along scan lines Li on the xy plane orthogonal to the z direction.

In the three-dimensional scan of this example, the B-scan for the scan lines L1 to L320 is executed four times in any order. The four B-scans for the scan lines Li are called repetition scans. The order of repetition of the four times for the scan lines Li may be set freely. For example, the four scans may be performed continuously, or a B-scan for other scan lines may be performed between the four scans.

The scan lines L1 to L320 are classified into sets of five according to their arrangement order. Each of the 64 sets obtained by this classification is called a unit, and a scan for each unit is called a unit scan. The unit scan includes four B-scans (repetition) for each of the five scan lines. That is, the unit scan includes twenty B-scans.

The image forming device 231 classifies data collected by applying such a scan pattern into data sets for each scan line Li (time-series data). Here, the data set includes four pieces of B-scan data corresponding to four times of repetition. Each of the four pieces of B-scan data is data collected by one B-scan for the scan line Li.

The image forming device 231 forms a motion contrast image corresponding to each scan line Li based on the data set corresponding to the scan line Li. The motion contrast image corresponding to each scan line Li is a two-dimensional angiographic image representing a B-scan plane including the scan line Li (longitudinal section).

The process of forming a motion contrast image is performed similarly as in OCT-angiography in the related art. As described above, in this example, the data set corresponding to the scan line Li includes four pieces of B-scan data. Each piece of B-scan data is data collected in one B-scan for the scan line Li.

First, the image forming device 231 forms a normal OCT image based on each piece of B-scan data. The OCT image is a B-scan image including 320 pieces of A-scan image data. Thus, four B-scan images corresponding to the scan lines Li are obtained.

Next, the image forming device 231 identifies an image region that changes between the four B-scan images. This process includes, for example, a process of obtaining the difference between different B-scan images. Each B-scan image is luminance image data (intensity image data) representing the morphology of the fundus oculi Ef, and it is considered that the image region corresponding to the portion other than blood vessel is substantially unchanged. On the other hand, considering that the backscattering contributing to the interference signal randomly changes due to the blood flow, the image region in which a change occurs between the four B-scan images (for example, the pixels in which the difference is not zero or the pixels in which the difference is equal to or greater than a predetermined threshold) can be estimated as a blood vessel region.

The image forming device 231 gives a predetermined pixel value to the pixels in the identified blood vessel region. The pixel value may be, for example, a relatively high luminance value (expressed bright and white when displayed) or a pseudo-color value. It is also possible to identify the blood vessel region using Doppler OCT or image processing, similarly to other techniques in the related art.

By such processing, 320 two-dimensional angiographic images corresponding to the 320 scan lines L1 to L320 are obtained. The image forming device 231 arranges the 320 two-dimensional angiographic images according to the arrangement of the 320 scan lines L1 to L320. This process includes, for example, a process of arranging (embedding) the 320 two-dimensional angiographic images in a single three-dimensional coordinate system in accordance with the arrangement order and the arrangement interval (spacing) of the 320 scan lines L1 to L320. That is, stack data on the 320 two-dimensional angiographic images corresponding to the arrangement of the 320 scan lines L1 to L320 can be formed. The stack data is an example of an image representing a three-dimensional distribution of blood vessels of the fundus oculi Ef (three-dimensional angiographic image). The image forming device 231 can also form volume data (voxel data) by performing interpolation or the like on the stack data.

The processing for forming the angiographic images from the collected data is not limited to the above example, and angiographic images can be formed using any known technique.

The image forming device 231 can process a three-dimensional image such as volume data or stack data. For example, the image forming device 231 can apply rendering to the three-dimensional image. Examples of the rendering method include volume rendering, maximum intensity projection (MIP), minimum intensity projection (MinIP), surface rendering, and multiplanar reformation (MPR). The image forming device 231 can construct projection data and a shadow gram by projecting at least a part of a three-dimensional image in the z direction (A-line direction, depth direction).

The image forming device 231 can execute any analysis and image processing. For example, the image forming device 231 can apply segmentation to a two-dimensional cross-sectional image or a three-dimensional image. The segmentation is a process of identifying a partial region in an image. In this example, an image region corresponding to a predetermined tissue of the fundus oculi Ef can be identified.

In OCT-angiography, the image forming device 231 can construct any two-dimensional angiographic image and/or any pseudo-three-dimensional angiographic image from a three-dimensional angiographic image. For example, the data processor 230 can construct a two-dimensional angiographic image representing any cross section of the fundus oculi Ef by applying multiplanar reformation to a three-dimensional angiographic image.

The image forming device 231 can apply segmentation to a three-dimensional angiographic image to identify an image region corresponding to a predetermined tissue of the fundus oculi Ef, and project the identified image region in the z direction to construct a shadow gram (en-face angiographic image). Examples of the en-face angiographic image include an en-face image corresponding to any depth region of the fundus Ef (for example, the shallow retina, the deep retina, the capillary lamina of choroid, the sclera, or the like), and an en-face image corresponding to a predetermined tissue of the fundus Ef (for example, the inner limiting membrane, the nerve fiber layer, the ganglion cell layer, the inner reticular layer, the inner nuclear layer, the outer reticular layer, the outer nuclear layer, the outer limiting membrane, the retinal pigment epithelium, the Bruch's membrane, the choroid, the choroid-scleral boundary, the sclera, a part of any of these, a combination of at least two of these, or the like).

The image forming device 231 may include, for example, an image processing processor and an image analysis processor in addition to the above-described image forming processor. The image forming processor is implemented by cooperation between hardware including circuitry and image forming software. The image processing processor is implemented by cooperation between hardware including circuitry and image processing software. The image analysis processor is implemented by cooperation between hardware including circuitry and image analysis software. In the present specification, “image data” and “image” based thereon may be regarded as the same. In addition, a site of the subject eye E and an image representing the region may be regarded as the same.

{Grade Evaluation Apparatus 500}

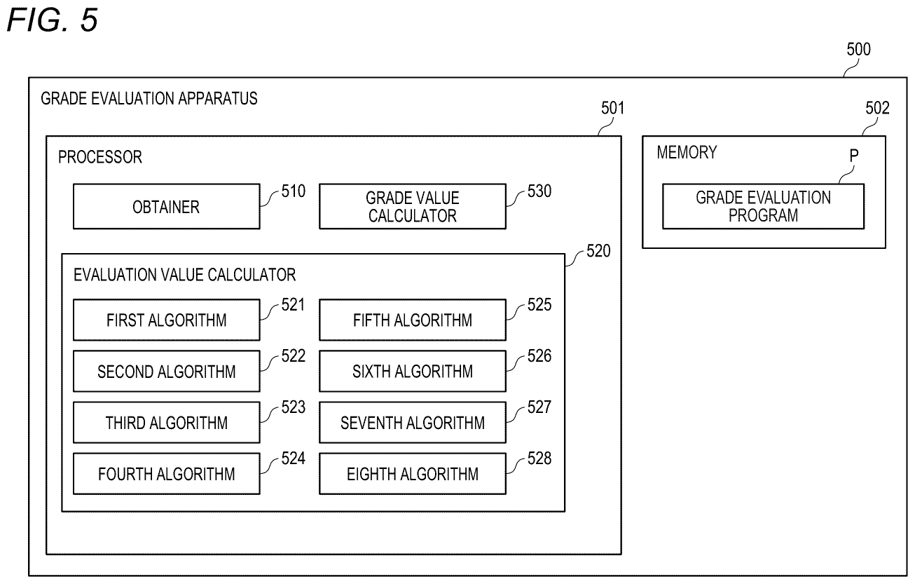

FIG. 5 is a schematic diagram illustrating an example of a configuration of the grade evaluation apparatus 500 according to the illustrative embodiment.

The grade evaluation apparatus 500 includes a processor 501 and a memory 502. The processor 501 is configured with a central processing unit (CPU), a graphic processing unit (GPU), an application specific integrated circuit (ASIC), a programmable logic device (for example, a simple programmable logic device (SPLD), a complex programmable logic device (CPLD), or a field programmable gate array (FPGA), and performs various processes and controls in cooperation with the memory 502. Specifically, the processor 501 functionally implements an obtainer 510, the evaluation value calculator 520, and the grade value calculator 530 by referring to a grade evaluation program P stored in the memory 502 and executing the program. The grade evaluation program P may be recorded in various recording media other than the memory 502, for example.

The memory 502 includes, for example, a random access memory (RAM) as a work memory used for the processing of the processor 501, and a read only memory (ROM) for storing a program that defines the processing of the processor 501. The RAM temporarily stores data generated or obtained by the processor 501. The program that defines the processing of the processor 501 is written in the ROM.

The memory 502 may store a blood vessel distribution image. The blood vessel distribution image is an image representing the distribution of blood vessels in the eyeball obtained by OCT-angiography. The blood vessel distribution image may be an image generated or stored by, for example, the control device 210, the OCT device 100, or the data processor 230 illustrated in FIG. 3.

The obtainer 510 obtains a blood vessel distribution image, which is an image representing the distribution of blood vessels in the eyeball obtained by OCT-angiography. The blood vessel distribution image obtained by the obtainer 510 may be an image stored in the memory 502. The blood vessel distribution image obtained by the obtainer 510 may be an image obtained from an external apparatus viewed from the grade evaluation apparatus 500. When a blood vessel distribution image is obtained from an external apparatus, the grade evaluation apparatus 500 may include a communication interface for connecting to an external apparatus or a communication network such as the Internet connected to the external apparatus.

The evaluation value calculator 520 calculates a plurality of evaluation values based on a plurality of criteria from the blood vessel distribution image obtained by the obtainer 510.

In the present illustrative embodiment, a plurality of algorithms are implemented in the evaluation value calculator 520. For example, a first algorithm 521, a second algorithm 522, a third algorithm 523, a fourth algorithm 524, a fifth algorithm 525, a sixth algorithm 526, a seventh algorithm 527, and an eighth algorithm 528 are implemented in the evaluation value calculator 520. The evaluation value calculator 520 calculates a plurality of evaluation values using these algorithms. The algorithm will be described in detail later. The algorithm implemented in the evaluation value calculator 520 is not limited to the eight algorithms described above.

The grade value calculator 530 calculates a grade value that is a value indicating the grade of the blood vessel distribution image based on the plurality of evaluation values calculated by the evaluation value calculator 520. Details of the grade value calculated by the grade value calculator 530 will be described later.

In addition, the grade evaluation apparatus 500 may further include an input unit for receiving information input from the user, an output unit for outputting information to the user, and the like. The grade evaluation apparatus 500 may be integrated with, for example, the control device 210, the OCT device 100, and the data processor 230 illustrated in FIG. 3.

The grade evaluation apparatus 500 may be incorporated in the ophthalmic imaging apparatus 1. That is, an ophthalmic imaging apparatus 1 capable of executing optical coherence tomography (OCT) may include the grade evaluation apparatus 500.

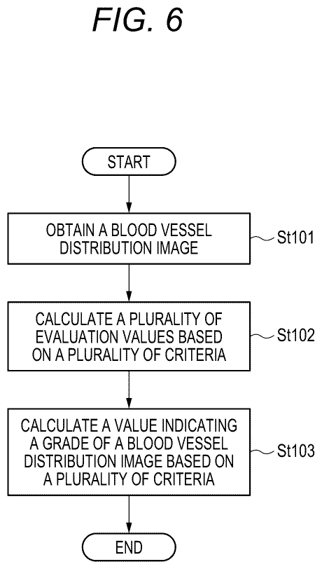

FIG. 6 is a flowchart illustrating an example of the operation of the grade evaluation apparatus 500 according to the illustrative embodiment.

The obtainer 510 obtains a blood vessel distribution image, which is an image representing the distribution of blood vessels in the eyeball obtained by OCT-angiography (St101).

The evaluation value calculator 520 calculates a plurality of evaluation values based on a plurality of criteria from the blood vessel distribution image obtained in step St101 (St102).

The grade value calculator 530 calculates a grade value that is a value indicating the grade of the blood vessel distribution image based on the plurality of evaluation values calculated in step St102 (St103).

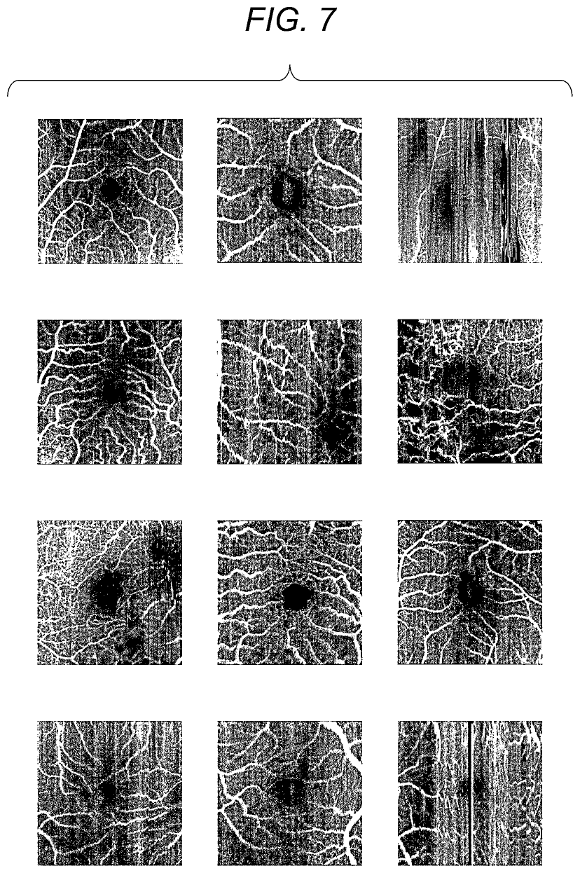

FIG. 7 is a diagram illustrating a blood vessel distribution image when the grade of the blood vessel distribution image is Poor (grade value=3). As illustrated, the blood vessel distribution image when the grade value is 3 includes many horizontal line artifacts, noise, and the like, which makes it difficult for the ophthalmologist or the diagnosis system to perform diagnosis based on the image.

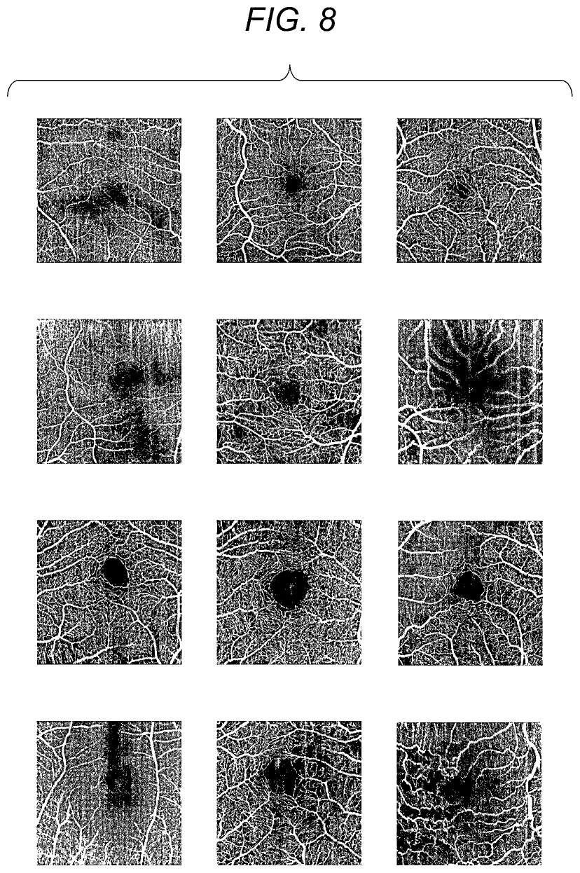

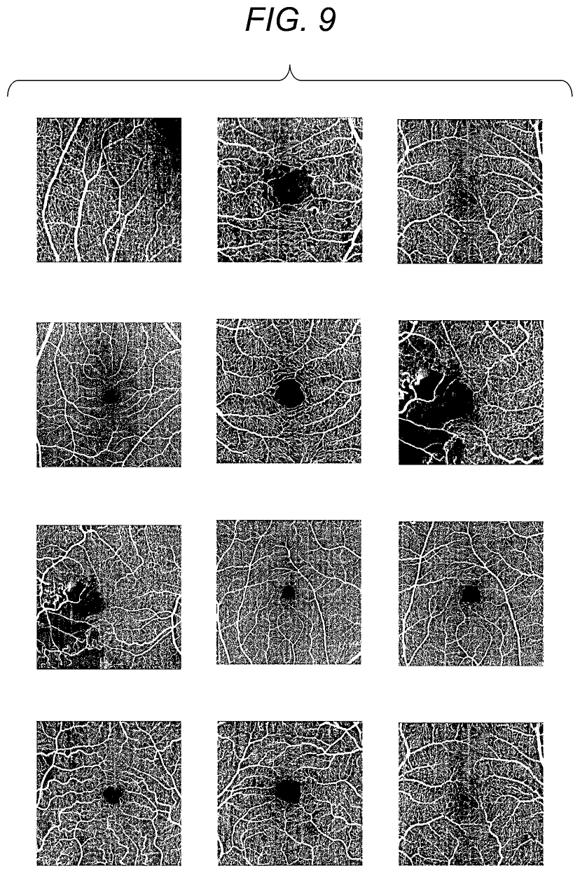

FIG. 8 is a diagram illustrating a blood vessel distribution image when the grade of the blood vessel distribution image is Average (grade value=2). FIG. 9 is a diagram illustrating a blood vessel distribution image when the grade of the blood vessel distribution image is Good (grade value=1). As illustrated, the blood vessel distribution image when the grade value is 1 or 2 has less horizontal line artifacts, noise, and the like than the blood vessel distribution image when the grade value is 3. The blood vessel distribution image when the grade value is 1 or 2 is suitable for the ophthalmologist or the diagnosis system to perform diagnosis based on the image.

The above has exemplified the case where the grade of the blood vessel distribution image is evaluated in three stages of 3 (Poor), 2 (Average), and 1 (Good), whereas the grade of the blood vessel distribution image is not limited to the three stages described above, and may be graded in two stages or five stages, for example.

There are a plurality of criteria for evaluating the grade of the blood vessel distribution image. However, it is difficult for a person such as an ophthalmologist to quantitatively determine the grade of the blood vessel distribution image while constantly considering a plurality of criteria. The grade evaluation apparatus 500 according to the illustrative embodiment of the present disclosure quantitatively evaluates the grade of the blood vessel distribution image based on a plurality of criteria.

{First Algorithm: EyeMotion}

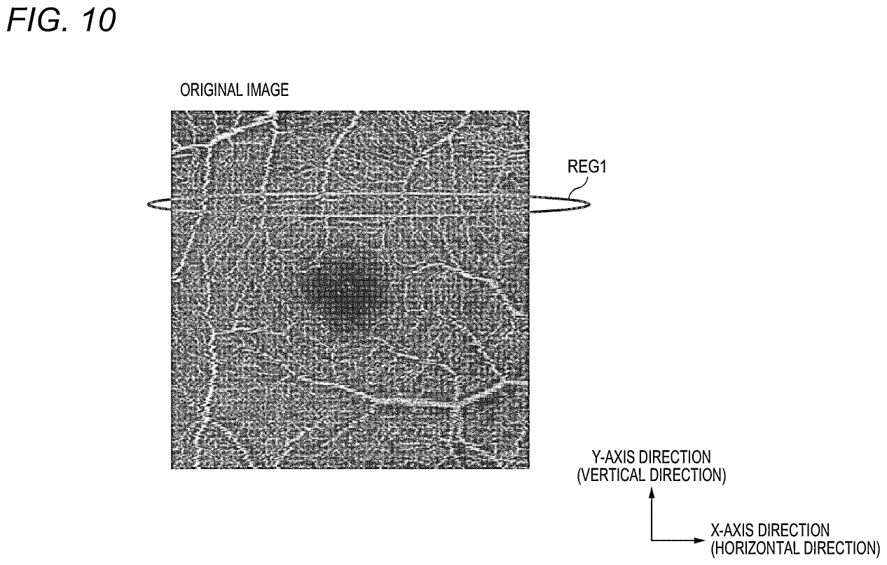

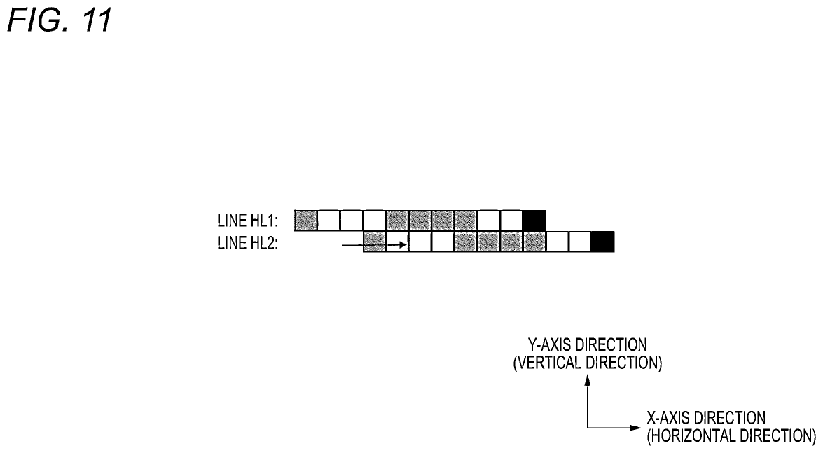

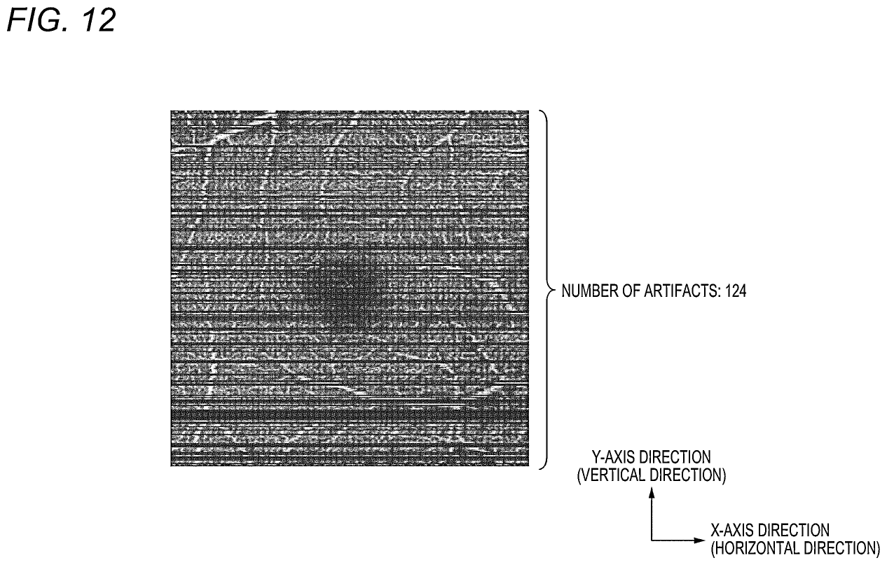

FIG. 10 is a schematic diagram of the first algorithm 521. FIG. 11 is a schematic diagram of the first algorithm 521. FIG. 12 is a schematic diagram of the first algorithm 521. The first algorithm 521 executed by the evaluation value calculator 520 will be described with reference to FIGS. 10 to 12.

The first algorithm 521 is an algorithm for calculating the number of artifacts generated in the blood vessel distribution image due to eye movement. FIG. 10 illustrates a blood vessel distribution image (original image) obtained by the obtainer 510. The Y-axis direction (vertical direction) and the X-axis direction (horizontal direction) in the image are both illustrated. In the original image, a portion with high brightness (white portion) is a blood vessel region, and a portion with low brightness (gray or black portion) is a region other than blood vessel (background region). As seen in a region REG1, the blood vessel distribution image may include a horizontal line artifact, in which the blood vessel is shifted in the horizontal direction due to eye movement. The first algorithm 521 is an algorithm for calculating the number of horizontal line artifacts as a first evaluation value.

As illustrated in FIG. 11, the lines HL1 and HL2 extend in the horizontal direction in the blood vessel distribution image. At a position where a horizontal line artifact occurs in the blood vessel distribution image, the correlation between the adjacent lines HL1 and HL2 becomes low due to the shift of the pixels in the horizontal direction. Therefore, the evaluation value calculator 520 calculates a value indicating the correlation between the adjacent lines HL1 and HL2. If the value indicating the correlation is lower than a predetermined value, the evaluation value calculator 520 determines that a horizontal line artifact is present at the position. The predetermined value is, for example, 0.3. The evaluation value calculator 520 counts the number of horizontal line artifacts in the blood vessel distribution image and calculates the number of horizontal line artifacts as a first evaluation value. In FIG. 12, the horizontal line artifacts detected by the above method are represented by black lines. The evaluation value calculator 520 calculates 124, which is the number of horizontal line artifacts, as a first evaluation value using the first algorithm 521. A smaller first evaluation value indicates a better grade of the blood vessel distribution image.

{Second Algorithm: BlackBand}

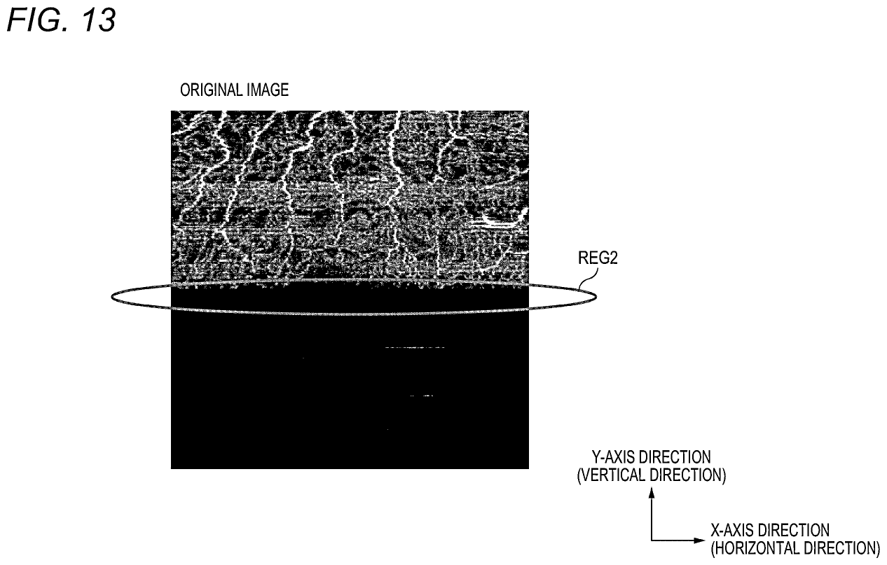

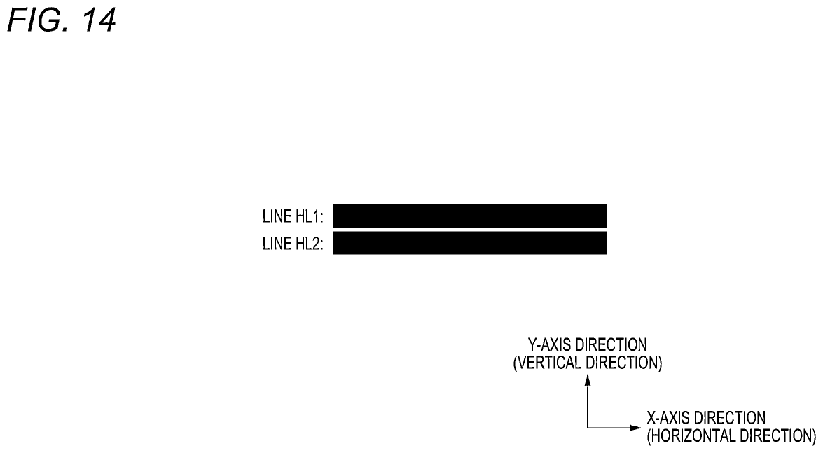

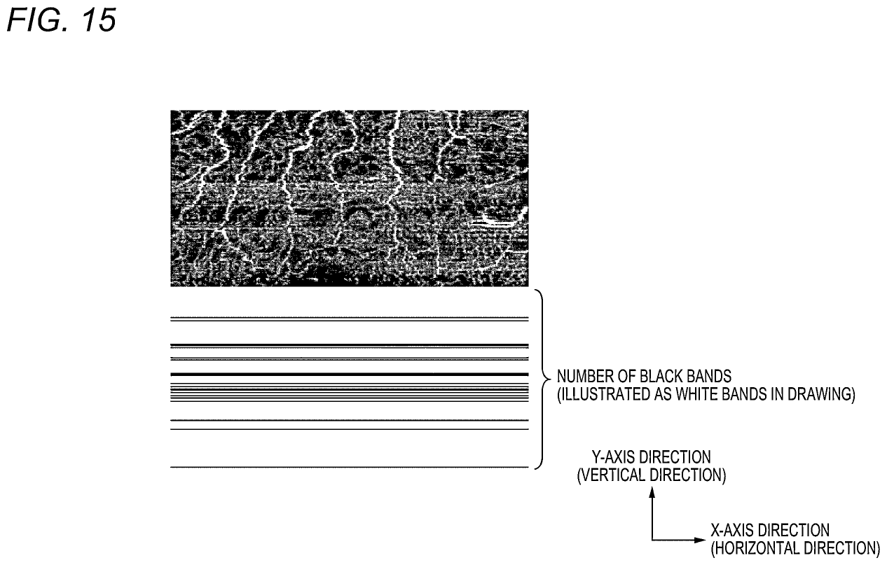

FIG. 13 is a schematic diagram of the second algorithm 522. FIG. 14 is a schematic diagram of the second algorithm 522. FIG. 15 is a schematic diagram of the second algorithm 522. The second algorithm 522 executed by the evaluation value calculator 520 will be described with reference to FIGS. 13 to 15.

FIG. 13 illustrates a blood vessel distribution image (original image) obtained by the obtainer 510. The Y-axis direction (vertical direction) and the X-axis direction (horizontal direction) in the image are both illustrated. In the original image, a portion with high brightness (white portion) is a blood vessel region, and a portion with low brightness (gray or black portion) is a region other than blood vessel (background region). As seen in a region REG2, the blood vessel distribution image may include black bands instead of blood vessels. The second algorithm 522 is an algorithm for calculating the number of black bands.

As illustrated in FIG. 14, the lines HL1 and HL2 extend in the horizontal direction in the blood vessel distribution image. In a portion where a black band occurs in the blood vessel distribution image, pixels having the same pixel value are continuous across a plurality of lines. Therefore, the evaluation value calculator 520 calculates a value indicating the correlation between the adjacent lines HL1 and HL2. If a value indicating the correlation is higher than the predetermined value, the evaluation value calculator 520 determines that a black band is present at the position. The predetermined value is, for example, 0.9. Then, the evaluation value calculator 520 counts the black bands in the blood vessel distribution image and calculates the number of black bands as a second evaluation value. For convenience of representation in the drawing, FIG. 15 displays a black band detected by the above-described method as a white band. A smaller second evaluation value indicates a better grade of the blood vessel distribution image.

{Third Algorithm: SNR}

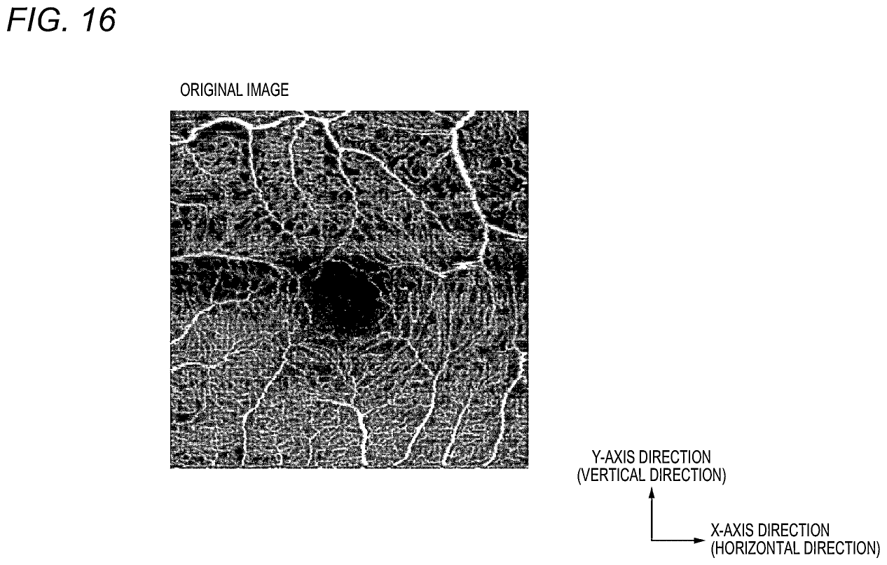

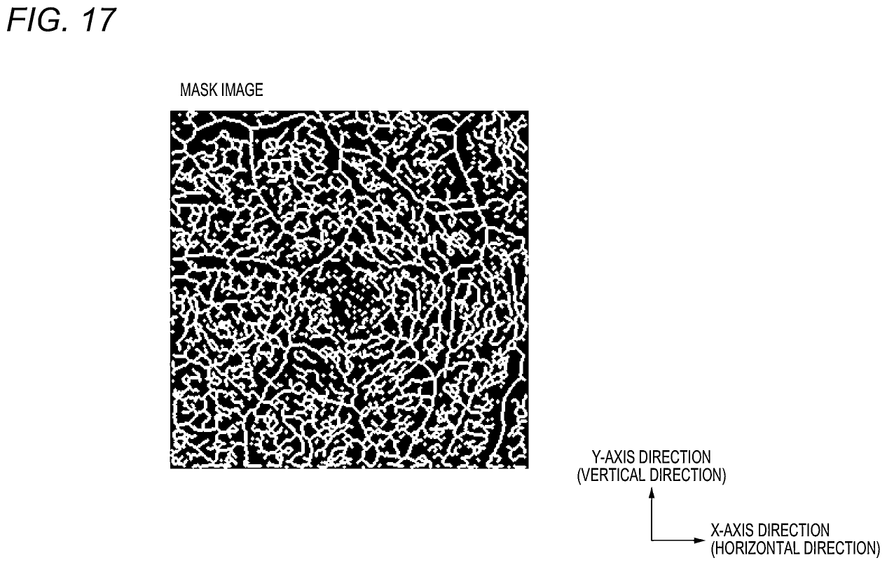

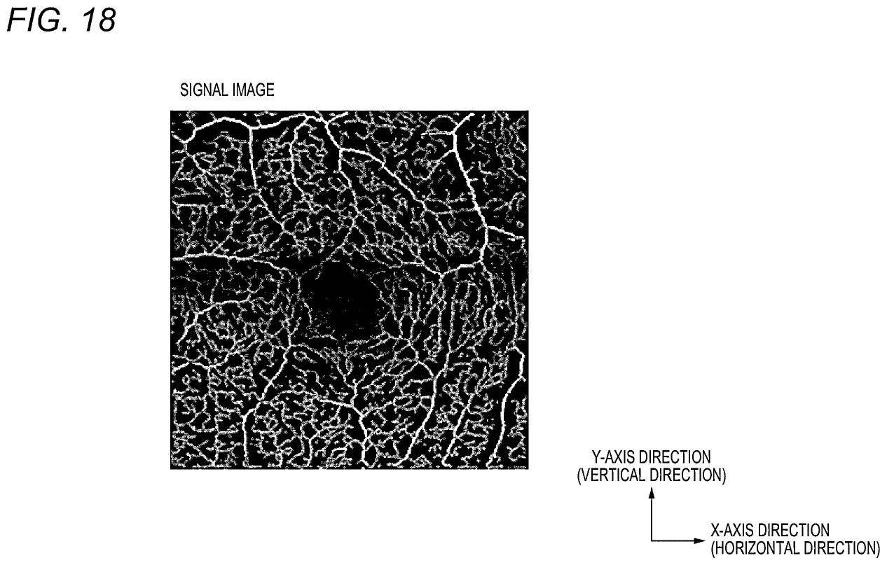

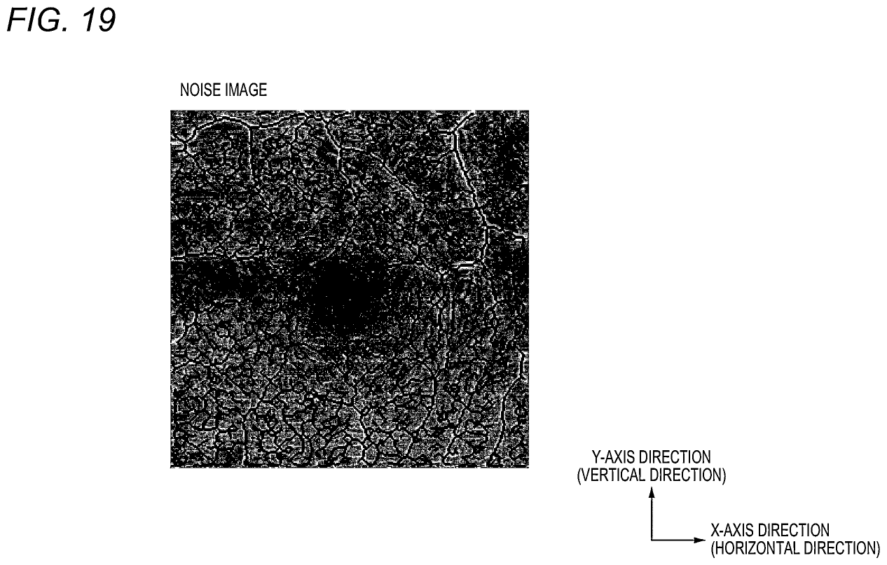

FIG. 16 is a schematic diagram of the third algorithm 523. FIG. 17 is a schematic diagram of the third algorithm 523. FIG. 18 is a schematic diagram of the third algorithm 523. FIG. 19 is a schematic diagram of the third algorithm 523. The third algorithm 523 executed by the evaluation value calculator 520 will be described with reference to FIGS. 16 to 19.

The third algorithm 523 is an algorithm for evaluating the contrast difference between blood vessel regions and regions other than blood vessel in the blood vessel distribution image. The signal-to-noise ratio (SNR) is used as a value representing the contrast difference. In the calculation of the SN ratio performed by the evaluation value calculator 520, a blood vessel region is treated as a signal, and a region other than blood vessel is treated as noise.

In order to calculate the SNR, a person skilled in the art may refer to, for example, the description of the following papers.
“Methods and algorithms for optical coherence tomography-based angiography: a review and comparison”, A. Zhang et al., Journal of Biomedical Optics 20(10), 100901 (October 2015)
“Image quality metrics for optical coherence angiography”, A. Lozzi et al., 1 Jul. 2015|Vol. 6, No. 7|BIOMEDICAL OPTICS EXPRESS 2435-2477

FIG. 16 illustrates a blood vessel distribution image (original image) obtained by the obtainer 510. The Y-axis direction (vertical direction) and the X-axis direction (horizontal direction) in the image are both illustrated. In the original image, a portion with high brightness (white portion) is a blood vessel region, and a portion with low brightness (gray or black portion) is a region other than blood vessel (background region). The evaluation value calculator 520 generates, from the original image, an image corresponding to the blood vessel regions and an image corresponding to the regions other than blood vessel. In order to generate these two types of images, the evaluation value calculator 520 performs image processing on the original image to create a mask image.

FIG. 17 illustrates the mask image generated by the evaluation value calculator 520 based on the original image. In order to generate the mask image, the evaluation value calculator 520 performs various image processing on the original image, such as adaptive binarization or deletion of objects of a predetermined size or smaller.

Next, the evaluation value calculator 520 multiplies the original image and the mask image. Thus obtained is a signal image as illustrated in FIG. 18 in which the blood vessel regions in the original image are extracted.

On the other hand, the evaluation value calculator 520 generates an inverted mask image in which the value of the binarized mask image (see FIG. 17) is inverted. The inverted mask image is an image obtained by replacing black and white in the mask image illustrated in FIG. 17. The evaluation value calculator 520 multiplies the original image and the inverted mask image. Thus obtained is a noise image as illustrated in FIG. 19 in which the regions other than blood vessel in the original image are extracted. The noise image is also referred to as a background image.

Subsequently, the evaluation value calculator 520 calculates the SNR based on the signal image and the noise image according to the following equation.

$$\begin{matrix}[ Formula 1 ] & \\ SNR = \frac{{μ}_{s} - {μ}_{b}}{\sqrt{{σ}_{s}^{2} + {σ}_{b}^{2}}} & ( 1 )\end{matrix}$$

In the above formula, μₛ means the average value of the pixel values of the signal image. σₛ means the standard deviation of the pixel values of the signal image. μ_{b} means the average value of the pixel values of the noise image. σ_{b} means the standard deviation of the pixel values of the noise image.

The evaluation value calculator 520 calculates the SNR as a third evaluation value using the third algorithm 523. A larger third evaluation value indicates a better grade of the blood vessel distribution image.

{Fourth Algorithm: Edge Sharpness}

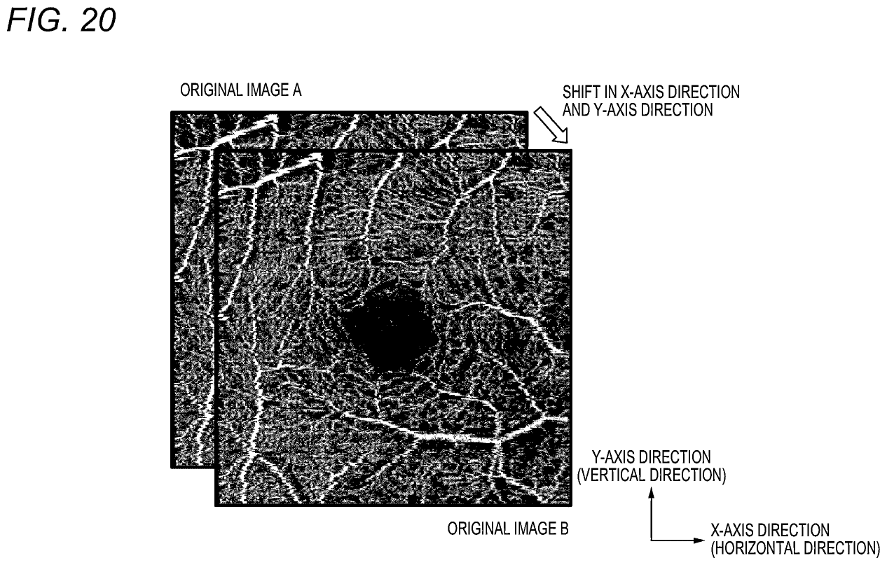

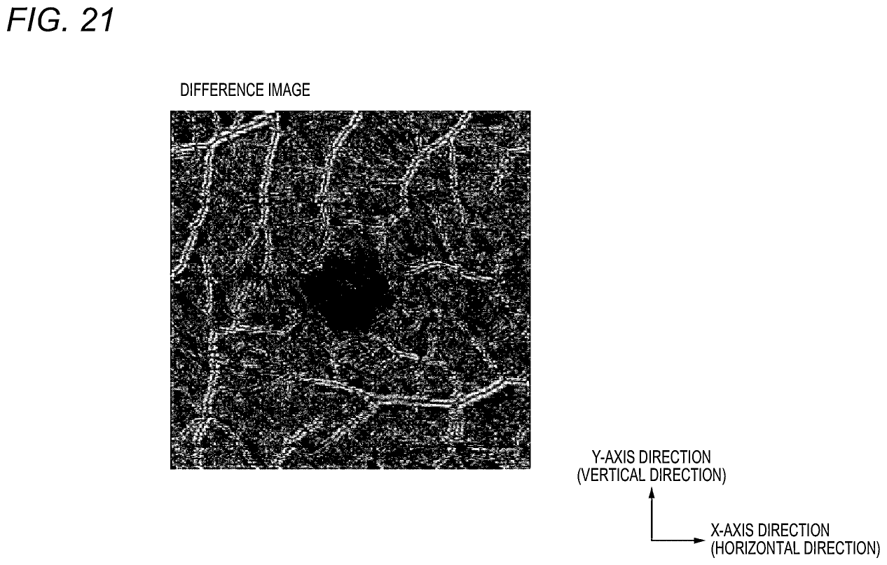

FIG. 20 is a schematic diagram of the fourth algorithm 524. FIG. 21 is a schematic diagram of the fourth algorithm 524. The fourth algorithm 524 executed by the evaluation value calculator 520 will be described with reference to FIGS. 20 and 21.

The fourth algorithm 524 is an algorithm for evaluating the sharpness of the blood vessel appearing in the blood vessel distribution image. In FIG. 20, two blood vessel distribution images (original images) obtained by the obtainer 510 are superimposed.

The evaluation value calculator 520 duplicates the original image obtained by the obtainer 510 to generate two original images. For convenience of description, the first original image is referred to as an original image A. The second original image is referred to as an original image B. The Y-axis direction (vertical direction) and the X-axis direction (horizontal direction) in the image are both illustrated. In the two original images, a portion with high brightness (white portion) is a blood vessel region, and a portion with low brightness (gray or black portion) is a region other than blood vessel (background region).

The evaluation value calculator 520 shifts the original image B by predetermined distances along the X axis and the Y axis of the image. The evaluation value calculator 520 superimposes the original image A and the original image B after the shift as illustrated in FIG. 20. Next, the evaluation value calculator 520 calculates the difference absolute value of the pixel values for the region where the two original images A and B overlap each other. For example, the evaluation value calculator 520 calculates the value of |a−b| for the coordinates (p, q), where a is the pixel value at the coordinates (p, q) of the original image A and b is the pixel value at the coordinates (p, q) of the original image B after the shift. An image defined by the absolute difference value (hereinafter referred to as a difference image) is illustrated in FIG. 21.

The evaluation value calculator 520 calculates an average value for each pixel included in the difference image. A larger average value indicates a higher sharpness of the blood vessel. That is, the evaluation value calculator 520 sets the pixel average value of the difference image as a fourth evaluation value. A larger fourth evaluation value indicates a better grade of the blood vessel distribution image.

{Fifth Algorithm: Connectivity}

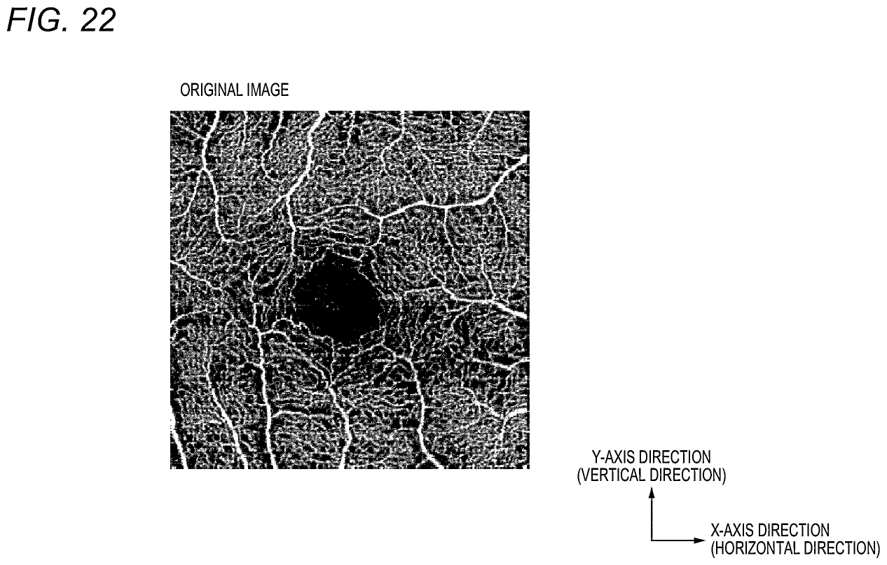

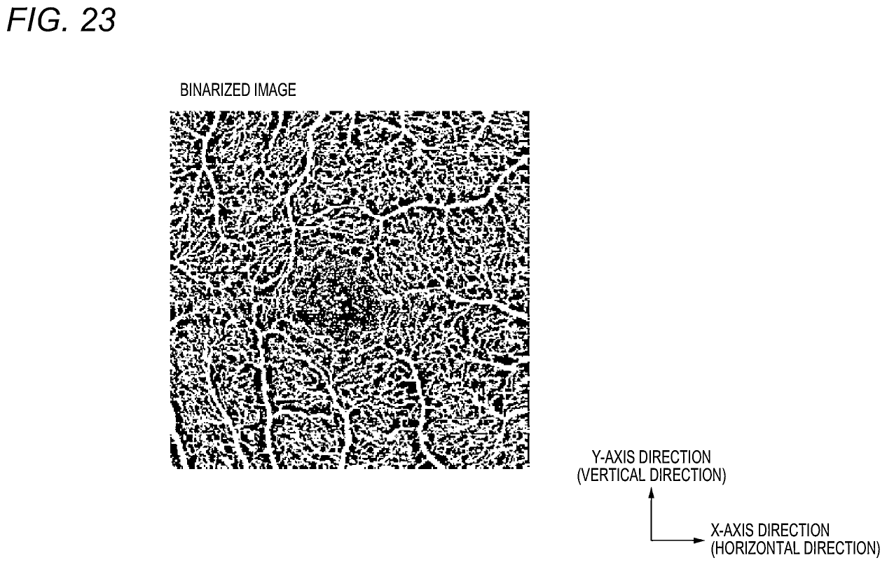

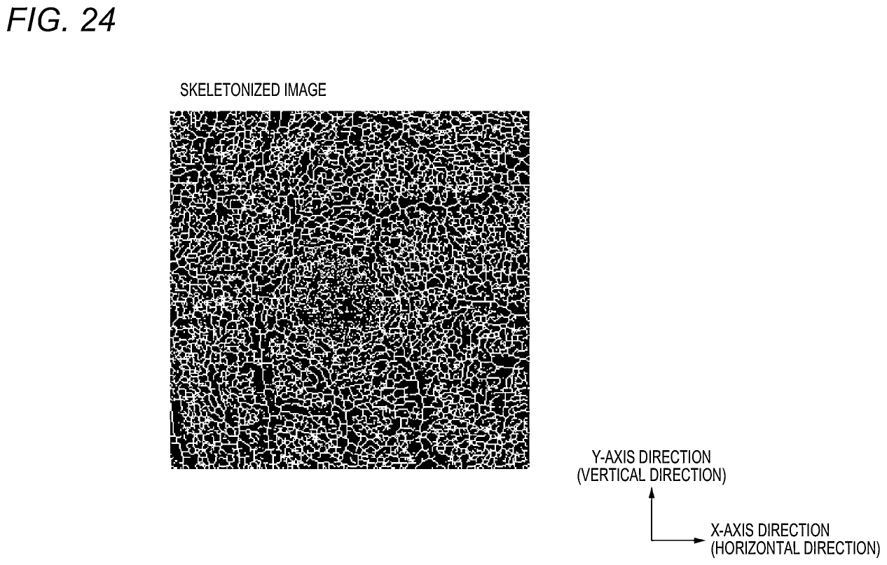

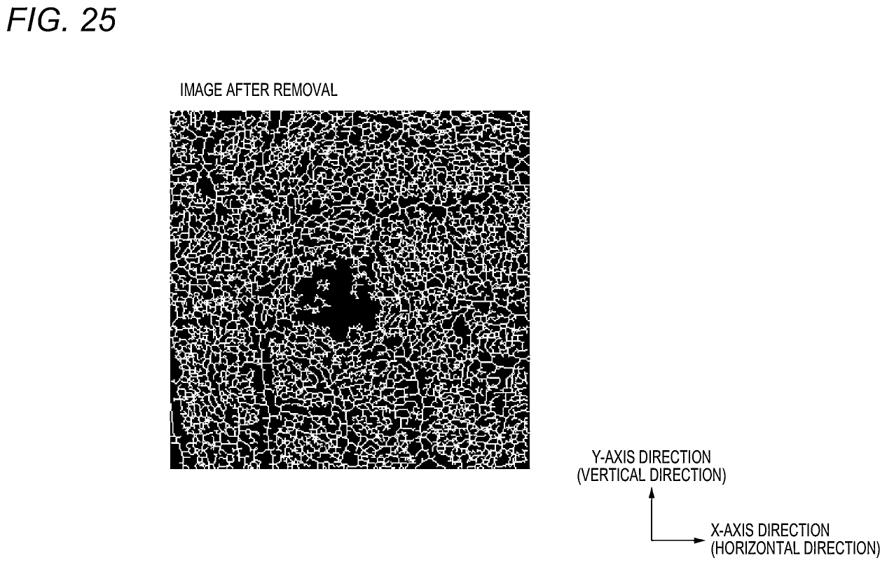

FIG. 22 is a schematic diagram of the fifth algorithm 525. FIG. 23 is a schematic diagram of the fifth algorithm 525. FIG. 24 is a schematic diagram of the fifth algorithm 525. FIG. 25 is a schematic diagram of the fifth algorithm 525. The fifth algorithm 525 executed by the evaluation value calculator 520 will be described with reference to FIGS. 22 to 25.

The fifth algorithm 525 is an algorithm for evaluating the degree of coupling of the blood vessels appearing in the blood vessel distribution image. The degree of coupling of blood vessels is a value indicating the degree of coupling of blood vessels in the blood vessel distribution image. The degree of coupling of the blood vessels is interpreted as low if the blood vessels appearing in the blood vessel distribution image are finely fragmented. The degree of coupling of the blood vessels is interpreted as high if the blood vessels appearing in the blood vessel distribution image are connected long. The degree of coupling of blood vessel can be calculated in the following manner with reference to, for example, “Split-spectrum amplitude-decorrelation angiography with optical coherence tomography”, Y. Jia et al., 13 Feb. 2012/Vol. 20, No. 4/OPTICS EXPRESS 4710-4725.

FIG. 22 illustrates a blood vessel distribution image (original image) obtained by the obtainer 510. The Y-axis direction (vertical direction) and the X-axis direction (horizontal direction) in the image are both illustrated. In the original image, a portion with high brightness (white portion) is a blood vessel region, and a portion with low brightness (gray or black portion) is a region other than blood vessel (background region). The evaluation value calculator 520 binarizes the original image.

FIG. 23 illustrates a binarized image obtained by binarizing the original image. The evaluation value calculator 520 skeletonizes the width of each blood vessel region (white portion in the drawing) appearing in the binarized image to the width of one pixel to generate a skeletonized image.

FIG. 24 illustrates a skeletonized image obtained by skeletonizing the width of the blood vessel region in the binarized image. The evaluation value calculator 520 removes the objects whose number of pixels corresponding to the object is equal to or less than a predetermined threshold from the objects appearing in the skeletonized image. The regions from which the objects are removed in the image are treated as regions other than blood vessel (background region). The predetermined threshold is, for example, 30. The image after the object removal (image after removal) is illustrated in FIG. 25.

The evaluation value calculator 520 calculates the ratio of the number of pixels of the blood vessel region in the image after removal as a fifth evaluation value. At this time, a predetermined number or more of pixels (for example, 30 pixels or more) are coupled in the regions remaining as blood vessels in the image. That is, a larger ratio of the number of pixels of the blood vessel region indicates that blood vessels having a higher degree of coupling appear in the original image. A larger fifth evaluation value indicates a better grade of the blood vessel distribution image.

{Sixth Algorithm: NoiseDetection1}

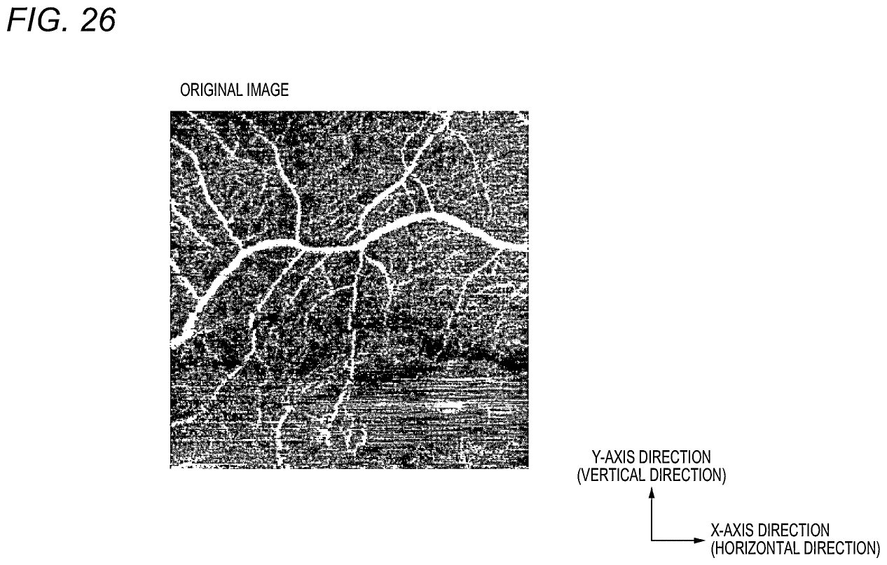

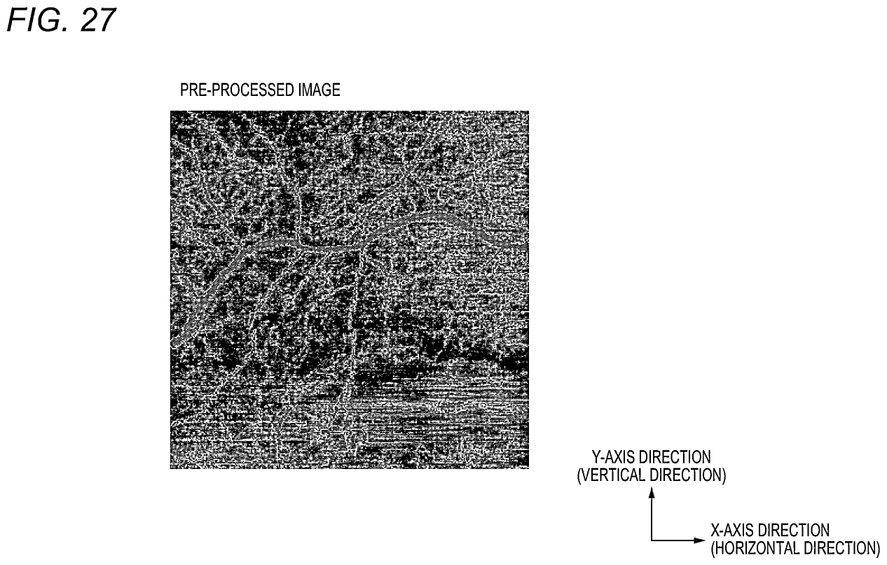

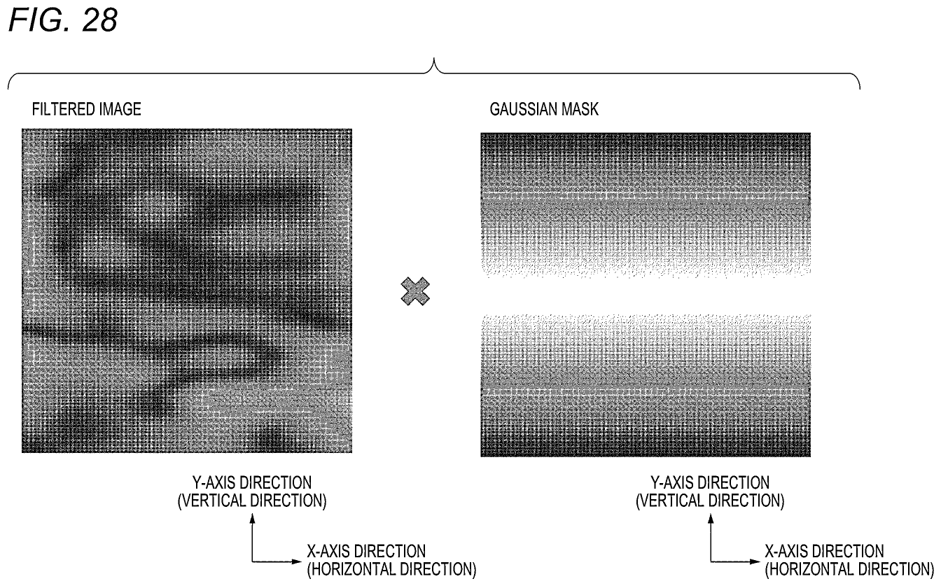

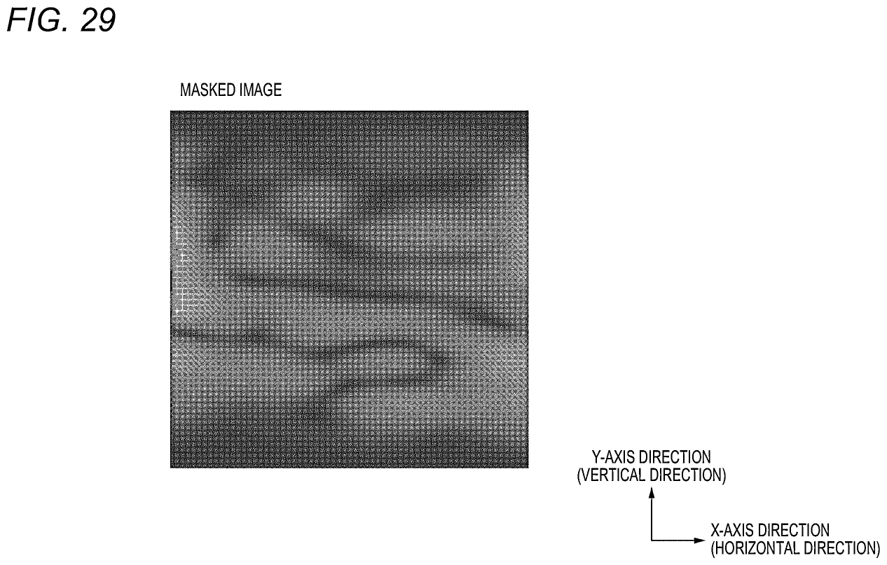

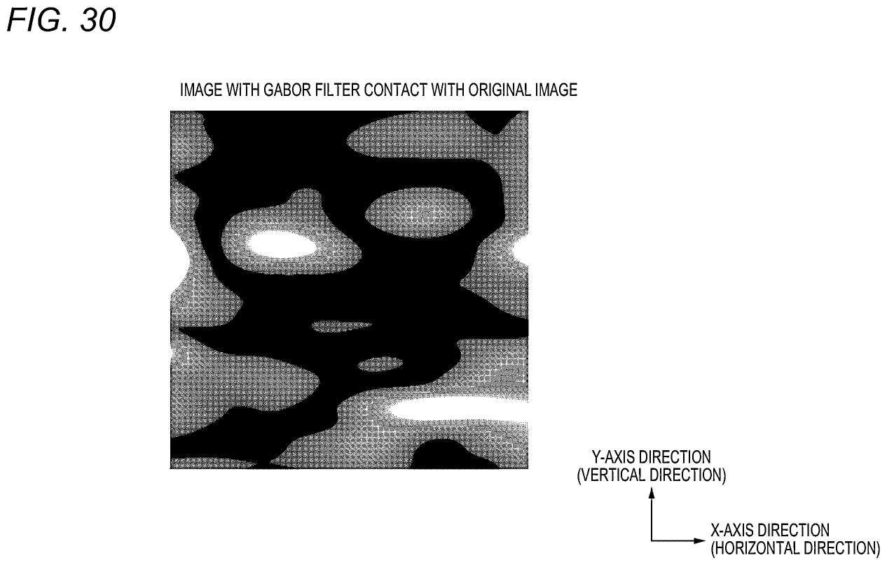

FIG. 26 is a schematic diagram of the sixth algorithm 526. FIG. 27 is a schematic diagram of the sixth algorithm 526. FIG. 28 is a schematic diagram of the sixth algorithm 526. FIG. 29 is a schematic diagram of the sixth algorithm 526. FIG. 30 is a schematic diagram of the sixth algorithm 526. The sixth algorithm 526 executed by the evaluation value calculator 520 will be described with reference to FIGS. 26 to 30.

The sixth algorithm 526 is an algorithm for detecting a horizontal line artifact generated as noise in the blood vessel distribution image. FIG. 26 illustrates a blood vessel distribution image (original image) obtained by the obtainer 510. The Y-axis direction (vertical direction) and the X-axis direction (horizontal direction) in the image are both illustrated. In the original image, a portion with high brightness (white portion) is a blood vessel region, and a portion with low brightness (gray or black portion) is a region other than blood vessel (background region).

In order to detect a horizontal line artifact included in the blood vessel distribution image, the evaluation value calculator 520 applies a Gabor filter. By applying the Gabor filter, the horizontal line artifacts can be detected because horizontal noise is conspicuous. However, if the Gabor filter is directly applied to the original image, the blood vessels extending in the horizontal direction are also detected as horizontal line artifacts as illustrated in FIG. 30. The evaluation value calculator 520 first pre-processes the original image to prevent the blood vessels extending in the horizontal direction, which are not originally noise, from being detected as horizontal line artifacts.

FIG. 27 illustrates an image obtained by pre-processing the original image (pre-processed image). The processing contents of the pre-processing are as follows, for example. The evaluation value calculator 520 calculates the pixel average value of the entire original image. The evaluation value calculator 520 extracts the regions having high signal intensity (blood vessel regions) in the original image. This extraction may be performed, for example, by extracting only the pixels whose signal intensities are equal to or greater than a predetermined threshold. Next, the evaluation value calculator 520 changes the value of each pixel having high signal intensity included in the extracted region to the calculated pixel average value. According to the pre-processing described above, the pixels corresponding to the blood vessel region has a signal intensity corresponding to the pixel average value of the original image, and thus are not detected as horizontal line artifacts after applying the Gabor filter described later.

The evaluation value calculator 520 applies the Gabor filter to the pre-processed image. FIG. 28 illustrates an image after applying the Gabor filter (filtered image). Here, consider a situation when a person such as an ophthalmologist evaluates the grade of the blood vessel distribution image. In this case, it is considered that the noise existing in the central portion of the blood vessel distribution image has a larger influence on the grade evaluation than the noise existing in the outer portion of the blood vessel distribution image. Therefore, the evaluation value calculator 520 performs processing for changing the weight of the detected noise between the central portion and the outer portion of the blood vessel distribution image. More specifically, the evaluation value calculator 520 multiplies the filtered image by a Gaussian mask. The Gaussian mask is illustrated in FIG. 28. FIG. 29 illustrates an image after applying the Gaussian mask to the filtered image (masked image).

The evaluation value calculator 520 calculates the noise average value of the entire masked image as a sixth evaluation value. A smaller sixth evaluation value indicates a better grade of the blood vessel distribution image.

{Seventh Algorithm: NoiseDetection2}

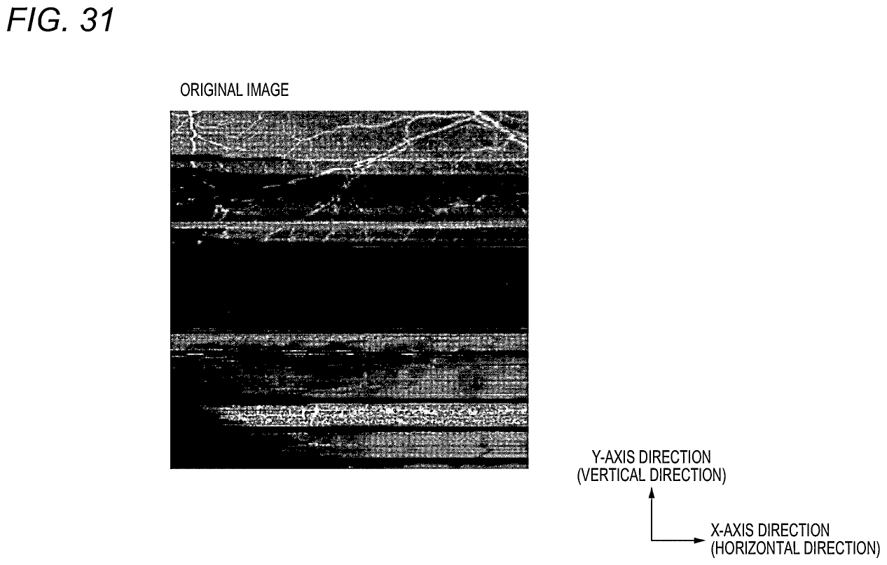

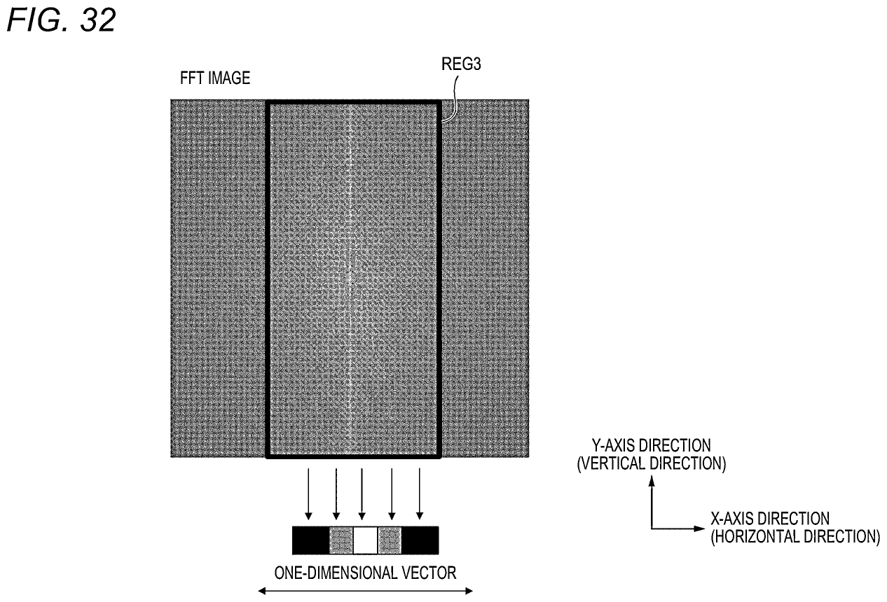

FIG. 31 is a schematic diagram of the seventh algorithm 527. FIG. 32 is a schematic diagram of the seventh algorithm 527. The seventh algorithm 527 executed by the evaluation value calculator 520 will be described with reference to FIGS. 31 and 32.

The seventh algorithm 527 is an algorithm for detecting a horizontal line artifact generated as noise in the blood vessel distribution image. FIG. 31 illustrates a blood vessel distribution image (original image) obtained by the obtainer 510. The Y-axis direction (vertical direction) and the X-axis direction (horizontal direction) in the image are both illustrated. In the original image, a portion with high brightness (white portion) is a blood vessel region, and a portion with low brightness (gray or black portion) is a region other than blood vessel (background region). The evaluation value calculator 520 performs fast Fourier transform (FFT) on the original image. FIG. 32 illustrates an image after the fast Fourier transform (FFT image).

When many horizontal line artifacts are included in the original image, one vertical white line appears at the center of the FFT image. Therefore, the evaluation value calculator 520 adds up the pixel values included in a predetermined region REG3 in the FFT image in the vertical direction (Y-axis direction), and generates a one-dimensional vector in which a plurality of total values are arranged in the horizontal direction (X-axis direction). Here, the region REG3 includes a central portion in the horizontal direction (X-axis direction) in the FFT image. The region REG3 has a predetermined width. The region REG3 may be the region of the entire FFT image.

The evaluation value calculator 520 calculates a standard deviation SD for the plurality of total values included in the generated one-dimensional vector. If the vertical white line appears at the center of the FFT image, the signal intensity of the central portion of the one-dimensional vector is high, and the signal intensity of the end portion is low. If many horizontal line artifacts are included in the original image, the value of the standard deviation SD for the plurality of total values included in the one-dimensional vector increases. The evaluation value calculator 520 calculates the value of the standard deviation SD as a seventh evaluation value using the seventh algorithm 527. A smaller seventh evaluation value indicates a better grade of the blood vessel distribution image.

Here, the evaluation value calculator 520 may calculate 1/SD, which is the reciprocal of the value of the standard deviation SD, as the seventh evaluation value using the seventh algorithm 527. In this case, a larger value of the seventh evaluation value indicates a better grade of the blood vessel distribution image.

{Eighth Algorithm: LocalContrast}

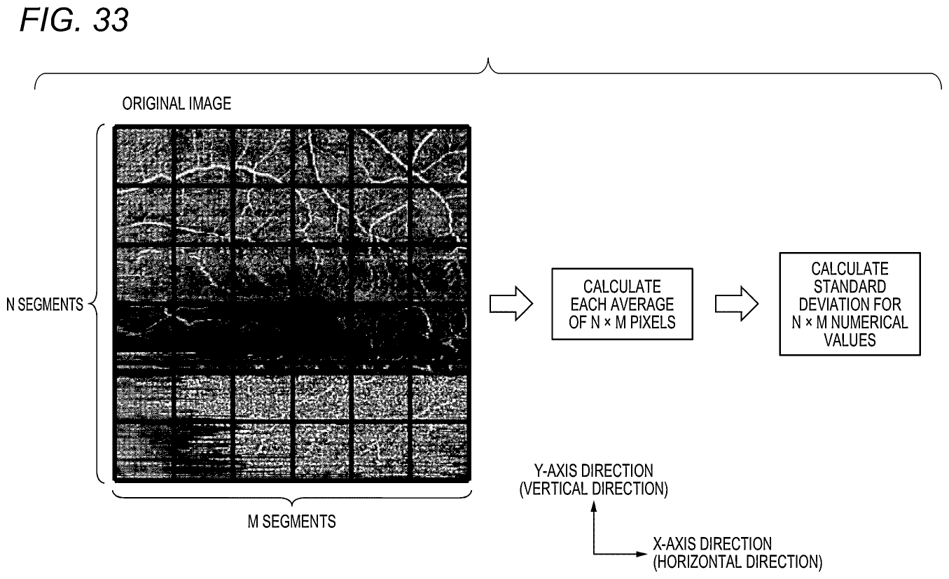

FIG. 33 is a schematic diagram of the eighth algorithm 528. The eighth algorithm 528 is an algorithm for evaluating the local contrast of the blood vessel distribution image, that is, the level of variation in contrast. FIG. 33 illustrates a blood vessel distribution image (original image) obtained by the obtainer 510. The Y-axis direction (vertical direction) and the X-axis direction (horizontal direction) in the image are both illustrated. In the original image, a portion with high brightness (white portion) is a blood vessel region, and a portion with low brightness (gray or black portion) is a region other than blood vessel (background region).

The evaluation value calculator 520 divides the original image into a plurality of sub-images. In the illustrated example, the evaluation value calculator 520 generates N×M sub-images by dividing the original image into N sections in the vertical direction (Y-axis direction) and M sections in the horizontal direction (X-axis direction).

Next, the evaluation value calculator 520 calculates, for each sub-image, a pixel average value on the sub-image. In the illustrated example, the evaluation value calculator 520 calculates N×M pixel average values. If the blood vessel distribution image includes local contrasts, a variation occurs between the N×M pixel average values. Therefore, the evaluation value calculator 520 calculates the standard deviation of the N×M pixel average values as an eighth evaluation value. A smaller eighth evaluation value indicates a better grade of the blood vessel distribution image.

{Grade Determination of Blood Vessel Distribution Image Based on a Plurality of Evaluation Values}

As described above, the evaluation value calculator 520 calculates a plurality of evaluation values based on a plurality of criteria. Each criterion corresponds to one algorithm described above. Next, the grade value calculator 530 calculates the grade value of the blood vessel distribution image based on the plurality of evaluation values calculated in step St102 (St103). The grade value calculator 530 can calculate the grade value of the blood vessel distribution image using regression or a decision tree.

{Grade Determination of Blood Vessel Distribution Image Based on Regression}

First, grade determination based on regression will be described. In the present illustrative embodiment, a general logistic regression is exemplified as the regression mode.

The general formula of the logistic regression is as follows.

$$\begin{matrix}[ Formula 2 ] & \\ y = \frac{1}{1 + {e}^{- ( {b}_{0} + {b}_{1} {x}_{1} + … + {b}_{n} {x}_{n} )}} & ( 2 )\end{matrix}$$

In the above general formula, y is the objective variable of logistic regression. The grade value calculator 530 may calculate the objective variable y as the grade value. Here, n is a natural number of 2 or more. In addition, n is equal to or less than the number of criteria (the number of algorithms) that can be used by the evaluation value calculator 520. If eight algorithms from the first algorithm 521 to the eighth algorithm 528 are implemented in the evaluation value calculator 520 as illustrated in FIG. 5, n is 8 or less.

x₁ to xₙ are explanatory variables of the logistic regression. b₀ is an error term, and b₁ to bₙ are partial regression coefficients. Values calculated in advance are used as the error term and the partial regression coefficients. For example, the values of the error term and the partial regression coefficient may be stored in the memory 502 of the grade evaluation apparatus 500 illustrated in FIG. 5. The grade value calculator 530 may obtain the error term and the value of the partial regression coefficient from an external apparatus.

The grade value calculator 530 uses a value based on the evaluation value calculated by the evaluation value calculator 520 as the explanatory variable xᵢ, where i is natural numbers of 1 to n. For example, if the number of explanatory variables is five, the evaluation value calculator 520 may input a value based on the seventh evaluation value to the explanatory variable x₁, input a value based on the sixth evaluation value to the explanatory variable x₂, input a value based on the first evaluation value to the explanatory variable x₃, input a value based on the third evaluation value to the explanatory variable x₄, and input a value based on the eighth evaluation value to the explanatory variable x₅. The input example of the value described above is merely an example, and a person skilled in the art may appropriately determine which evaluation value is to be input to each explanatory variable.

The value based on the evaluation value may be the evaluation value itself. The value based on the evaluation value may be a value obtained by performing predetermined calculation on the evaluation value, such as the absolute value, the inverse number, the logarithm, or the square root of the evaluation value.

For example, as described above, the grade value calculator 530 calculates the value of the objective variable y using the values based on the evaluation values calculated by the evaluation value calculator 520 as the explanatory variables xᵢ. The value of y corresponds to the grade value calculated by the grade value calculator 530. As is clear from the above-described general formula (2), the value of y in the case of the logistic regression is a continuous value from 0 to 1. At this time, the grade of the blood vessel distribution image is for example, grade 1: Good if the value of y is larger than a predetermined first threshold. The value of y is grade 3: Poor if the value of y is smaller than a predetermined second threshold. The value of y is grade 2: Average if the value of y is a value between the predetermined first threshold and the predetermined second threshold. The first threshold is larger than the second threshold.

In the above example, five explanatory variables are used. However, the number of explanatory variables may be other than five, for example, eight. The type of regression used by the grade value calculator 530 is not limited to the above-described logistic regression, and regression other types may be used.

{Grade Determination of Blood Vessel Distribution Image Based on Decision Tree}

Next, grade determination based on a decision tree will be described. In the grade evaluation apparatus 500 according to the illustrative embodiment, the grade value calculator 530 uses a decision tree. The decision tree may be, for example, a classification tree for classifying the blood vessel distribution image into three classes. The three classes are grade 1: Good, grade 2: Average, and grade 3: Poor.

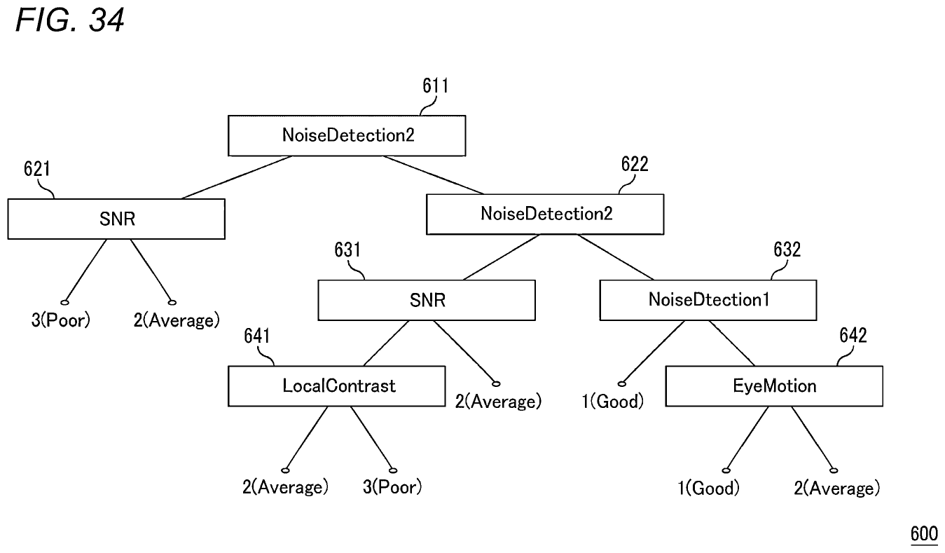

FIG. 34 is a conceptual diagram illustrating a decision tree 600 used by the grade evaluation apparatus 500 according to the illustrative embodiment. The first layer of the decision tree 600 has a node 611 as the root node. Edges extend from the node 611 to the nodes 621 and 622 in the second layer. Edges extend from the node 622 to the nodes 631 and 632 in the third layer. An edge extends from the node 631 to the node 641 in the fourth layer. An edge extends from the node 632 to the node 642 in the fourth layer.

Condition determination using explanatory variables is performed in each node in the decision tree 600. The grade value calculator 530 calculates the grade value by the decision tree 600 using the values based on the plurality of evaluation values calculated by the evaluation value calculator 520 as explanatory variables.

The grade value calculator 530 starts processing from the node 611 which is the root node. In the node 611, the grade value calculator 530 branches the subsequent process to the node 621 or the node 622 using the value of 1/SD, which is the seventh evaluation value according to the seventh algorithm (NoiseDetection2). The grade value calculator 530 is configured to perform processing of the node 621 if, for example, the value of 1/SD is less than a predetermined value, and to perform processing of the node 622 if the value of 1/SD is equal to or greater than the predetermined value.

In the node 621, the grade value calculator 530 uses the value of SNR which is the third evaluation value related to the third algorithm (SNR). The grade value calculator 530 calculates 3 (Poor) as the grade value if, for example, the value of SNR is less than a predetermined value. The grade value calculator 530 calculates 2 (Average) as the grade value if the value of SNR is equal to or greater than the predetermined value.

In the node 622, the grade value calculator 530 branches the subsequent process to the node 631 or the node 632 using the value of 1/SD, which is the seventh evaluation value according to the seventh algorithm (NoiseDetection2). The grade value calculator 530 performs processing of the node 631 if, for example, the value of 1/SD is less than a predetermined value, and performs processing of the node 632 if the value of 1/SD is equal to or greater than the predetermined value.

In the node 631, the grade value calculator 530 uses the value of SNR which is the third evaluation value related to the third algorithm (SNR). The grade value calculator 530 performs processing of the node 641 if, for example, the value of SNR is less than a predetermined value. The grade value calculator 530 calculates 2 (Average) as the grade value if the value of SNR is equal to or greater than the predetermined value.

In the node 632, the grade value calculator 530 uses the noise average value of the entire masked image, which is the sixth evaluation value according to the sixth algorithm (NoiseDetection1). The grade value calculator 530 calculates 1 (Food) as the grade value if, for example, the noise average value is less than a predetermined value. The grade value calculator 530 performs processing of the node 642 if, for example, the noise average value is equal to or greater than a predetermined value.

In the node 641, the grade value calculator 530 uses the value of the standard deviation of the N×M pixel average values, which is the eighth evaluation value according to the eighth algorithm (LocalContrast). The grade value calculator 530 calculates 2 (Average) as the grade value if the value of the standard deviation of the N×M pixel average values is less than a predetermined value. The grade value calculator 530 calculates 3 (Poor) as the grade value if the value of the standard deviation of the N×M pixel average values is equal to or greater than the predetermined value.

In the node 642, the grade value calculator 530 uses the number of horizontal line artifacts, which is the first evaluation value related to the first algorithm (EyeMotion). The grade value calculator 530 calculates 1 (Food) as the grade value if, for example, the number of horizontal line artifacts is less than a predetermined value. The grade value calculator 530 calculates 2 (Average) as the grade value if the number of horizontal line artifacts is equal to or greater than the predetermined value.

For example, as described above, the grade value calculator 530 of the grade evaluation apparatus 500 according to the illustrative embodiment of the present disclosure calculates the grade value by the decision tree 600 using the values based on the plurality of evaluation values as explanatory variables. The decision tree 600 is a classification tree for classifying the blood vessel distribution image into three classes: Good, Average, and Poor. The grade value calculated by the grade value calculator 530 takes three types of values (1, 2, and 3) corresponding to the three classes, respectively.

The node-edge structure in the decision tree 600 may be determined in advance. For example, information defining the node-edge structure in the decision tree 600 may be stored in the memory 502 of the grade evaluation apparatus 500.

The node-edge structure in the decision tree 600 is not limited to that illustrated in FIG. 26. A person skilled in the art can appropriately change, for example, the depth of the hierarchy of the decision tree, the value based on which evaluation value is used as the explanatory variable in each node, the branch condition from each node to the next node (a threshold for comparison with the value based on the evaluation value, or the like), and the class to be classified.

The node-edge structure in the decision tree may be obtained by machine learning using learning data with the plurality of evaluation values as explanatory variables and the grade value as the objective variable. As the algorithm for performing machine learning, decision tree learning as a technique in the related art may be used.

As described above, the blood vessel distribution image grade evaluation apparatus 500 according to the illustrative embodiment includes the obtainer 510, the evaluation value calculator 520, and the grade value calculator 530. The obtainer 510 obtains a blood vessel distribution image, which is an image representing the distribution of blood vessels in the eyeball obtained by OCT-angiography. The evaluation value calculator 520 calculates a plurality of evaluation values from the blood vessel distribution image based on a plurality of criteria. The grade value calculator 530 calculates a grade value which is a value indicating the grade of the blood vessel distribution image based on the plurality of evaluation values. Accordingly, the grade evaluation apparatus 500 can quantitatively evaluate the grade of the blood vessel distribution image obtained by OCT-angiography based on the plurality of criteria.

The grade value calculator 530 calculates a grade value by logistic regression using values based on a plurality of evaluation values as explanatory variables. Accordingly, the grade value calculator 530 can calculate the grade value as a continuous value. Therefore, a more accurate grade evaluation can be implemented by appropriately adjusting the threshold for distinguishing the grade.

The grade value calculator 530 calculates the grade value by a decision tree using values based on the plurality of evaluation values as explanatory variables. Accordingly, the grade value calculator 530 can calculate an appropriate grade value by flexibly using a plurality of evaluation values.

The decision tree is a classification tree for classifying the blood vessel distribution image into three classes, and the grade value takes three types of values respectively corresponding to the three classes. Accordingly, the grade evaluation apparatus 500 can evaluate the grade of the blood vessel distribution image in three stages: Poor, Average, and Good.

The decision tree has a node-edge structure obtained by machine learning using learning data with the plurality of evaluation values being explanatory variables and the grade value being an objective variable. Thus, the decision tree can be improved by machine learning. Therefore, the grade evaluation apparatus 500 can perform the grade evaluation with higher accuracy.

The evaluation value calculator 520 calculates an evaluation value indicating the number of horizontal line artifacts in the blood vessel distribution image. Accordingly, the grade evaluation apparatus 500 can evaluate the grade of the blood vessel distribution image in consideration of the number of horizontal line artifacts.

The evaluation value calculator 520 calculates an evaluation value indicating the number of black bands in the blood vessel distribution image. Accordingly, the grade evaluation apparatus 500 can evaluate the grade of the blood vessel distribution image in consideration of the number of black bands.

The evaluation value calculator 520 calculates an evaluation value indicating an SNR in which a blood vessel in the blood vessel distribution image is treated as a signal and a region other than blood vessel is treated as noise. Accordingly, the grade evaluation apparatus 500 can evaluate the grade of the blood vessel distribution image in consideration of the SNR.

The evaluation value calculator 520 calculates an evaluation value indicating sharpness of a blood vessel in the blood vessel distribution image. Accordingly, the grade evaluation apparatus 500 can evaluate the grade of the blood vessel distribution image in consideration of the sharpness of the blood vessel in the blood vessel distribution image.

The evaluation value calculator 520 calculates an evaluation value indicating the degree of coupling of blood vessels in the blood vessel distribution image. Accordingly, the grade evaluation apparatus 500 can evaluate the grade of the blood vessel distribution image in consideration of the degree of coupling of the blood vessels in the blood vessel distribution image.

The evaluation value calculator 520 calculates an evaluation value indicating noise in a horizontal line direction in the blood vessel distribution image. Accordingly, the grade evaluation apparatus 500 can evaluate the grade of the blood vessel distribution image in consideration of the noise in the horizontal line direction in the blood vessel distribution image.

The evaluation value calculator 520 calculates an evaluation value indicating a level of variation in contrast in the blood vessel distribution image. Accordingly, the grade evaluation apparatus 500 can evaluate the grade of the blood vessel distribution image in consideration of the level of variation in contrast in the blood vessel distribution image.

The ophthalmic imaging apparatus 1 capable of executing optical coherence tomography (OCT) includes the grade evaluation apparatus 500. Thus, the ophthalmic imaging apparatus 1 can not only image the subject eye, but also generate a blood vessel distribution image by performing OCT and quantitatively evaluate the grade of the blood vessel distribution image based on a plurality of criteria.

The program P causes the computer to function as the obtainer 510, the evaluation value calculator 520, and the grade value calculator 530. The obtainer 510 obtains a blood vessel distribution image, which is an image representing the distribution of blood vessels in the eyeball obtained by OCT-angiography. The evaluation value calculator 520 calculates a plurality of evaluation values from the blood vessel distribution image based on a plurality of criteria. The grade value calculator 530 calculates a grade value which is a value indicating the grade of the blood vessel distribution image based on the plurality of evaluation values. A non-transitory computer-readable storage medium (for example, the memory 502) records the program P. Accordingly, it is possible to quantitatively evaluate the grade of the blood vessel distribution image obtained by OCT-angiography based on the plurality of criteria.

A computer having the processor 501 executes a blood vessel distribution image grade evaluation method. In the grade evaluation method, the processor 501 calculates a plurality of evaluation values based on a plurality of criteria from a blood vessel distribution image which is an image representing a distribution of blood vessels in an eyeball obtained by OCT-angiography. The processor 501 calculates a grade value that is a value indicating the grade of the blood vessel distribution image based on the plurality of evaluation values. Accordingly, it is possible to quantitatively evaluate the grade of the blood vessel distribution image obtained by OCT-angiography based on the plurality of criteria.

Although various illustrative embodiments have been described above with reference to the drawings, it is needless to say that the present disclosure is not limited to such an example. It is apparent to those skilled in the art that various modification examples, modification examples, substitution examples, addition examples, deletion examples, and equivalent examples can be conceived within the scope described in the claims, and it is understood that these examples naturally belong to the technical scope of the present disclosure. In addition, the constituent elements in the various illustrative embodiments described above may be arbitrarily combined without departing from the scope of the invention.

Although various illustrative embodiments have been described above with reference to the drawings, the present invention is not limited to these examples. It is apparent to those skilled in the art that various modifications or modifications can be conceived within the scope described in the claims, and it is understood that the modifications or modifications naturally fall within the technical scope of the present invention. In addition, the components described in the above illustrative embodiments may be arbitrarily combined without departing from the scope of the invention.

The present disclosure is useful as a grade evaluation apparatus, an ophthalmic imaging apparatus, a non-transitory computer-readable storage medium, and a grade evaluation method for quantitatively evaluating the grade of a blood vessel distribution image obtained by OCT-angiography based on a plurality of criteria.

What is claimed is:

1. A grade evaluation apparatus for blood vessel distribution image, comprising:
a control device configured to:
obtain a blood vessel distribution image, the blood vessel distribution image representing a distribution of blood vessels in an eyeball obtained by OCT-angiography;
calculate a plurality of evaluation values from the blood vessel distribution image based on a plurality of criteria; and
calculate a grade value based on the plurality of evaluation values, the grade value indicating a grade of the blood vessel distribution image.

2. The grade evaluation apparatus according to claim 1, wherein in the calculating of the grade value, the control device is configured to calculate the grade value by logistic regression using values based on the plurality of evaluation values as explanatory variables.

3. The grade evaluation apparatus according to claim 1, wherein in the calculating of the grade value, the control device is configured to calculate the grade value by a decision tree using values based on the plurality of evaluation values as explanatory variables.

4. The grade evaluation apparatus according to claim 3,
wherein the decision tree is a classification tree for classifying the blood vessel distribution image into three classes, and
wherein the grade value takes three types of values respectively corresponding to the three classes.

5. The grade evaluation apparatus according to claim 3, wherein the decision tree has a node-edge structure obtained by machine learning using learning data with the plurality of evaluation values being explanatory variables and the grade value being an objective variable.

6. The grade evaluation apparatus according to claim 1, wherein in the calculating of the evaluation value, the control device is configured to calculate an evaluation value indicating the number of horizontal line artifacts in the blood vessel distribution image.

7. The grade evaluation apparatus according to claim 1, wherein in the calculating of the evaluation value, the control device is configured to calculate an evaluation value indicating the number of black bands in the blood vessel distribution image.

8. The grade evaluation apparatus according to claim 1, wherein in the calculating of the evaluation value, the control device is configured to calculate an evaluation value indicating a signal-to-noise ratio (SNR) in which a blood vessel in the blood vessel distribution image is treated as a signal and a region other than blood vessel is treated as noise.

9. The grade evaluation apparatus according to claim 1, wherein in the calculating of the evaluation value, the control device is configured to calculate an evaluation value indicating sharpness of a blood vessel in the blood vessel distribution image.

10. The grade evaluation apparatus according to claim 1, wherein in the calculating of the evaluation value, the control device is configured to calculate an evaluation value indicating a degree of coupling of blood vessels in the blood vessel distribution image.

11. The grade evaluation apparatus according to claim 1, wherein in the calculating of the evaluation value, the control device is configured to calculate an evaluation value indicating noise in a horizontal line direction in the blood vessel distribution image.

12. The grade evaluation apparatus according to claim 1, wherein in the calculating of the evaluation value, the control device is configured to calculate an evaluation value indicating a level of variation in contrast in the blood vessel distribution image.

13. An ophthalmic imaging apparatus for executing optical coherence tomography (OCT), comprising the grade evaluation apparatus according to claim 1.

14. A non-transitory computer-readable storage medium storing a computer program readable by a computer, the computer program, when executed by the computer, causing the computer to perform operations comprising:
obtaining a blood vessel distribution image, the blood vessel distribution image representing a distribution of blood vessels in an eyeball obtained by OCT-angiography;
calculating a plurality of evaluation values from the blood vessel distribution image based on a plurality of criteria; and
calculating a grade value based on the plurality of evaluation values, the grade value indicating a grade of the blood vessel distribution image.

15. A grade evaluation method for blood vessel distribution image by a computer including a processor, the grade evaluation method comprising causing the processor to perform:
calculating a plurality of evaluation values based on a plurality of criteria from a blood vessel distribution image, the blood vessel distribution image representing a distribution of blood vessels in an eyeball obtained by OCT-angiography; and
calculating a grade value based on the plurality of evaluation values, the grade value indicating a grade of the blood vessel distribution image.
